(19) United States
(12) Patent Application Publication
(10) Pub. No.: US 2004/0254444 A1
(43) Pub. Date: Dec. 16, 2004

(54) Use of MRI to screen normal risk, asymptomatic individuals for breast cancer

(76) Inventor: Craig Bittner, Scottsdale, AZ (US)

Correspondence Address: PATTON BOGGS LLP, 8484 WESTPARK DRIVE, SUITE 900, MCLEAN, VA, 22102, US

(21) Appl. No.: 10/460,797

(22) Filed: Jun. 13, 2003

(51) Int. Cl. A61B 5/05

(52) U.S. Cl. 600/410

(57) ABSTRACT

Methods for screening normal risk, asymptomatic individuals for breast cancer are provided, which may enable early diagnosis of breast cancer. The methods may involve the use of a contrast agent while screening normal risk, asymptomatic individuals using MRI technology. The methods may involve use of MRI spectroscopy while screening individuals using MRI technology. Also provided are methods of detecting breast cancer in normal risk, asymptomatic individuals, which include screening such individuals using MRI technology.

### FIELD OF THE INVENTION

The present invention relates to methods of screening normal risk, asymptomatic individuals for breast cancer, and to methods of detecting breast cancer in normal risk, asymptomatic individuals using Magnetic Resonance Imaging (MRI) technology.

### BACKGROUND OF THE INVENTION

Breast cancer is a major source of cancer morbidity and mortality in individuals. Early detection is the most important factor to survival, with a survival rate of 96%, if found early.

Breast Self Examination (BSE) is the most common method of breast cancer detection. About two-thirds of cancers are detected by palpation. Such sensitivity of BSE is related to significant changes in mechanical properties of tissues in the course of breast cancer development. The BSE is widely advised and taught to individuals as a means of pre-clinical testing and contributes significantly to early cancer detection. A major fraction of the occurrences of breast cancer is first detected by individuals themselves, who bring any perceived problems to the attention of their physicians. The usefulness of palpatory self-examination as a pre-clinical test is well proven by a wealth of data. The major drawback of this method is that only large size nodules are detectable by palpation. An average examiner, including individuals conducting self-examinations, does not reliably detect lesions until they approach 2 cm in diameter, which may represent a significantly advanced stage of cancer. Even a highly experienced examiner is able to detect a nodule only over 1 cm in diameter, which still may correspond to a considerably advanced cancer.

Currently, the most widely used clinical diagnostic method is mammography. Efforts to reduce mortality via screening mammography have been successful with definite improvement in survival, particularly in individuals over 50 years old. Mammography may however, miss breast cancer in its early stages when it is most amenable to treatment and most likely to be cured. Mammography is also disadvantageous in that it is limited in individuals who have breast implants, and is not as accurate in younger individuals whose breast tissue tends to be denser. Furthermore, mammography exposes individuals to ionizing radiation that may increase their risk of developing breast cancer. Mammography also requires significant compression of the breast tissue that many individuals find painful, leading them to avoid having mammography.

These methods do not provide an adequate, sufficiently accurate early detection of breast cancer in normal risk, asymptomatic individuals.

### SUMMARY OF THE INVENTION

The present invention provides methods of screening normal risk, asymptomatic individuals for breast cancer and methods of detecting breast cancer in normal risk, asymptomatic individuals, which includes screening such individuals using MRI technology. According to embodiments of the invention, the methods may involve the use of a contrast agent while screening normal risk, asymptomatic individuals using MRI technology. According to embodiments of the invention, these methods may also involve use of MRI spectroscopy while screening individuals using MRI technology.

The present invention also provides methods of detecting breast cancer in individuals, which include screening such individuals using MRI spectroscopy technology.

The present invention may enable more accurate and earlier diagnosis of breast cancer, particularly in individuals who are asymptomatic and have not been diagnosed as being at a high risk for breast cancer.

### BRIEF DESCRIPTION OF THE DRAWINGS

FIG. 1 is a flow chart representative of an algorithm showing assessment of diagnostic information obtained using MRI to screen normal risk, asymptomatic individuals for breast cancer.

FIGS. 2-3 are sample curves demonstrating relative enhancement over time for a benign nodule in the breast and for a cancer, respectively.

FIGS. 4-8 are screen captures of examples of how the control panel of the MRI may be set up for imaging protocols in accordance with methods of the invention.

FIGS. 9-21 depict sample MRI images of an individual who had a normal examination according to the methods of the present invention.

FIGS. 22-34 depict sample MRI images of an individual according to the methods of the present invention. This individual had one or more benign lesions in the breasts.

FIGS. 35-47 depict sample MRI images of an individual according to the methods of the present invention. This individual had breast cancer.

### DESCRIPTION OF THE INVENTION

This application relates to the following patent application filed concurrently herewith, which is hereby incorporated by reference in its entirety: “Use of MRI to Screen Individuals for Prostate Cancer,” (attorney docket # 011339.0106).

There is a need for an effective and more accurate method for the screening of normal risk, asymptomatic individuals for the presence of breast cancer. Availability of an easy to employ, more accurate methodology for regular testing will lead to vast improvement in early and accurate diagnosing with the lowering of the morbidity and mortality of breast cancer.

The present invention provides methods for screening normal risk, asymptomatic individuals for breast cancer using MRI technology. In particular, the methods include performing MRI on normal risk individuals who are asymptomatic for breast cancer; and determining from the MRI whether those individuals have indications of breast cancer.

The present invention also provides methods of detecting breast cancer in normal risk, asymptomatic individuals, which include screening normal risk individuals who are asymptomatic for breast cancer, where the screening includes performing MRI on the individuals; and determining from the MRI whether those individuals have indications of breast cancer.

Normal risk individuals are those who do not have a significant family history of breast cancer and have not otherwise been told by a physician that for whatever reason they have a high risk of acquiring breast cancer due to genetics, behavior or other characteristic.

Asymptomatic individuals are those who have neither detected a suspicious lump by BSE, mammography and/or ultrasound nor experienced any of the other symptoms of breast cancer including for example nipple discharge, breast pain or architectural distortion of the breast.

MRI has become an important non-invasive medical technique over the past decade. U.S. Pat. No. 6,411,837, which is hereby incorporated by reference, discloses a method for high-resolution magnetic resonance tomography of the female breast. U.S. Pat. No. 6,468,231, which is hereby incorporated by reference, discloses a method and device for detecting changes in mechanical and structural properties of breast tissue. U.S. Pat. No. 6,363,275, which is hereby incorporated by reference, discloses a device for detecting and for treating tumors using differential diagnosis.

MRI uses a strong direct current magnetic field in conjunction with tunable gradient magnetic fields to spatially control locations at which the net sum magnetic field reaches a preselected value. As the magnetic bias fields are varied spatially, a series of radio frequency (RF) pulses are applied. When the RF energy is at a resonance frequency of sample atoms of a particular species and surroundings, those sample atomic nuclei absorb the RF energy and are excited to a higher spin state. The excited spin state then decays to a lower energy state of excitation and the decay is accompanied by an emission of an RF pulse, known as “spin echo.” The RF of a nucleus and its resulting spin echo signal depend on a number of factors, including mass, density, dipole moment, relaxation frequency, as well as chemical bonding and electrostatic potential of its surroundings.

According to embodiments of the invention, the methods of the invention utilize MRI technology. MRI is performed on one breast individually or on both breasts simultaneously. Images of the breast or breasts are acquired using various MRI sequences, such as T1, T2 and Proton Weighted images of the breast. The images demonstrate the internal structure of the breast tissue and various specific MRI protocols can be used to accentuate the image characteristics of various components that may be present within the breast such as glandular tissue, vascular tissue, fat tissue, cysts, fibroadenomata or breast cancer. The images can be reviewed by a board-certified radiologist or other individual utilizing specialized medical workstations that help evaluate the image data to aid in the detection of breast cancer even at its earliest stages. The radiologist or other interpreting individual and workstations may be either on-site at the location where the MRIs are being performed, or they may be at a remote location to where the MRI images are sent.

According to embodiments of the invention, the disclosed methods may involve use of a contrast agent while screening normal risk, asymptomatic individuals using MRI technology. In particular, in order to enhance the contrast between tissues within an organism, one or more contrast agents may be introduced into an individual's body prior to MRI analysis. Contrast agents, which may be used in accordance with the present invention include suitable contrast agents known to those in the art, including for example, paramagnetic metal ions such as manganese, gadolinium and iron.

The large dipoles associated with paramagnetic ions, as compared to protons, perturbs the proton's relaxation time, T, as a function of distance between the ion and the proton, thus providing a strong differential signal which can be used for imaging. WO 02/13874, which is hereby incorporated by reference, discloses the use of metal complexes containing perfluoroalkyl as contrast agents in MRI for the representation of tumoral tissue. WO 01/82976, WO 00/16811 and WO 99/26535, which are also hereby incorporated by reference, disclose MRI contrast imaging agents for imaging cancer. These contrast agents may be used in accordance with the present invention.

The methods of the invention are novel in the way they examine the function of the breast tissue as well as its structure. Neither x-ray mammography nor ultrasound currently has this capability. Because breast cancer can often appear structurally the same as normal breast tissue, these tests are much less accurate. For a cancer to grow it must establish its own blood supply in a process called angiogenesis. In angiogenesis, the cancer will form new blood vessels that are unlike any normally found in the breasts of adults. In particular, these angiogenic blood vessels demonstrate abnormal physiologic function which is distinguishable from that of normal adult blood vessels found in non-cancerous tissue. Thus, according to embodiments of the invention, utilizing a special technique called Dynamic Enhancement Imaging (DEI), MRI can evaluate the breast tissue for the abnormal function of these angiogenic blood vessels.

In accordance with certain embodiments of the methods of the invention, possible sites of breast cancer in an individual's breast can be identified with the aid of MRI by means of a dynamic investigation in which the contrast enhancement behavior of the breast tissue is evaluated. The contrast agent may be introduced by injection into a vein in the arm of an individual (or introduced by other suitable means such as injection into a vein or artery elsewhere in the body) in a relatively small dose on the order of approximately 10-30 mL, approximately 15-25 mL, or approximately 15 mL. A secondary injection (or other form of administration) into the individual of a physiological saline solution or other suitable fluid of about the same volume as that of the contrast agent may also be used so that the contrast agent reaches the blood circulation as completely as possible.

According to embodiments of the invention, a baseline pre-contrast MRI of a single breast or of both breasts is performed, and then a contrast agent is administered in a dosage of approximately 0.07-0.13 mmol/kg, 0.085-0.115 mmol/kg, or approximately 0.1 mmol/kg of body weight of the individual. By performing a dynamically enhanced MRI with T1 weighting of the breast over a period of approximately 2 to 10 minutes, approximately 3 to 8 minutes, or approximately 5 minutes at specific intervals, such as intervals of approximately every 5 to 90 seconds, or approximately every 60 seconds, cancer from normal breast tissue can be differentiated with a very high degree of sensitivity and specificity. The dynamically enhanced MRI may be started within approximately 0-120 seconds or approximately 40 seconds from the time the contrast agent is started being injected. The contrast agent is administered at a bolus rate of approximately 1 to 3 cc/sec or approximately 2 cc/sec and subsequent MRI imaging through the breast is then acquired at approximately 5 second to 90 second intervals, or approximately 60 second intervals. By this method, the degree of percentage of MRI signal enhancement over time may be evaluated and a percentage of enhancement versus time curve plotted. Regions of cancer growth within the breast demonstrate a significantly higher degree of enhancement than surrounding normal breast tissue early after contrast agent administration and this degree of enhancement tends to decreases more quickly than that of the normal tissue. This difference in enhancement is a direct effect of the differing physiology of the cancer's angiogenic blood vessels.

Accordingly, the present invention also includes a method of screening a normal risk, asymptomatic individual for breast cancer, which includes obtaining a baseline pre-contrast MRI of one or both breasts of an individual, administering a contrast agent to the individual in an amount of approximately 0.07-0.13 mmol/kg, 0.085-0.115 mmol/kg, or approximately 0.1 mmol/kg body weight of the individual, performing a dynamically enhanced MRI with T1 weighting of the breast over a period of approximately 2 to 10 minutes, approximately 3 to 8 minutes, or approximately 5 minutes at specific intervals of approximately every 5 to 90 seconds, evaluating and possibly plotting a degree of percentage of MRI signal enhancement over time, and identifying any regions of possible cancer from that of normal breast tissue based on the degree of enhancement in addition to the structural characteristics of the tissues present in the MRI images.

T1 weighting is a description of any MRI protocol that accentuates the differing longitudinal magnetization relaxation of protons. MRI protocols can also be employed that accentuate the T2 weighting of various protons by accentuating their differing transverse magnetization characteristics or that accentuate the proton density of tissue by minimizing both the T1 and T2 weighting within the MRI protocols. These differences are achieved by altering the TR (Time of Repetition) and TE (Time of Echo) of the RF (radio frequency) pulses used to excite the protons to an excited spin state, and their behavior is determined by their inherent T1 and T2 relaxation (or decay) properties.

T2 weighted images and Proton Density weighted images may also be gathered in accordance with the present invention, which may be useful in identifying a structural abnormality within the breast tissue and in distinguishing cancer from benign tissue such as for example distinguishing a cancer from a fibroadenoma or a cyst. This information may be used in conjunction with the information obtained in the dynamic enhancement images such as plotting relative MRI signal enhancement over time for an area of structural abnormality seen in the non-dynamic enhanced images.

As with other methods of the invention, a board certified radiologist or other individual identifies any regions of possible cancer growth from normal breast tissue possibly utilizing specialized medical workstations that help to evaluate the information obtained in the dynamic contrast images and other MRI images. The radiologists and workstations may be either on-site at the location where the MRIs are being performed, or they may be at a remote location to where the MRI images are sent.

The contrast agent may include one or more suitable contrast agents, such as those described above, which include, but are not limited to paramagnetic metal ions, including for example, manganese, gadolinium and/or iron. According to these methods, the contrast agent may be administered at a bolus rate of approximately 1 to 3 cc/sec or approximately 2 cc/sec.

The contrast agent examinations may be conducted bilaterally, i.e., the methods of the invention may be performed on both breasts of an individual, and thereby both breasts examined substantially simultaneously. Performing the examinations simultaneously saves examination time, because given a unilateral examination, due to the use of the contrast agent, the second breast cannot be examined immediately after the first. Also, it aids in identifying possible cancer by allowing the direct comparison of the tissue within each breast. In addition, bilateral examination prevents the individual from being unduly stressed by a second injection. The MRI can ensue either in the transversal, the coronal or any other suitable slice orientation that allows both breasts to be imaged simultaneously.

According to embodiments of the invention, the disclosed methods may involve the use of MRI spectroscopy while screening individuals using MRI technology.

MRI spectroscopy can non-invasively obtain physiologic images and spectra, based on the relative concentrations of cellular chemicals and metabolites. MRI spectroscopy can provide physiologic information about the relative concentrations of metabolites such as, citrate, creatine, and choline within the breast tissue by measuring the specific resonances for the metabolites from small volumes of tissue throughout the breast. The amount of individual resonance present for each metabolite is related to the concentration of these metabolites and changes in these concentrations can be used to identify breast cancer from normal breast tissue and from non-cancerous changes of the breast tissue. For example, breast cancer will demonstrate significantly higher choline levels and significantly lower citrate levels as compared to normal breast tissues and benign changes in the breast. The ratio of certain metabolites (e.g., choline/citrate) in regions of cancer have minimal overlap with values of either normal breast tissue or benign breast lesions. Also, the physiologic information obtained from MRI spectroscopy may enable assessment of cancer aggressiveness.

Accordingly, the present invention provides methods of detecting breast cancer in individuals, which include screening individuals for breast cancer, where the screening includes performing MRI spectroscopy on one or more individuals; and determining from the MRI spectroscopy whether those individuals have indications of breast cancer.

The present invention further provides methods of screening individuals for breast cancer, which include obtaining a MRI spectroscopy of one or both breasts of an individual, determining relative concentrations of one or more metabolites within the breast tissue by measuring specific resonances of the metabolites from small volumes of tissue throughout the breast, determining ratios of the metabolites in the breast, and differentiating regions of cancer growth from normal breast tissue based, inter alia, on the ratios. The metabolites may include one or more of the following exemplary metabolites, citrate, creatine and choline, and other metabolites known to those skilled in the art. The individual may be a normal risk individual who is asymptomatic for breast cancer. Small volumes of tissue may include, for example, volumes of approximately 1 mm³ to 1 cm³ from various portions throughout the breast as determined by a skilled practitioner. Accordingly to these embodiments, one breast may be screened, or both breasts of an individual may be screened simultaneously.

The present methods of detecting breast cancer using MRI have significant advantages in the field of breast cancer screening, including e.g.: (1) the improved ability to detect early breast cancers; (2) the improved ability to differentiate between breast cancer and normal breast tissue; (3) the improved ability to differentiate between breast cancer and benign lesions in the breast; (4) the improved ability to differentiate between scar and recurrent breast cancer; (5) the improved detection of multifocal and/or multicentric breast cancers; (6) the improved ability to determine the extent of breast cancer present; (7) the improved ability to detect early breast cancer in individuals with breast implants; (8) the improved ability to detect breast cancer in individuals with breast implants while also evaluating the breast implants for abnormalities; and (9) the improved ability to detect early breast cancer in individuals with dense breasts.

Current breast cancer screening methods, such as breast self-examination and x-ray mammography, are disadvantageous in many respects. For example, they are not able to accurately detect early breast cancer and most abnormalities identified by these methods are later found to not be cancerous leading to unnecessary medical costs and procedures. X-ray mammography, which only obtains images of the breast based upon the differing x-ray densities of the tissues within the breast, is not highly effective in detecting early breast cancer nor in differentiating benign tissue from cancerous tissue. For individuals having breast implants and/or highly dense breast tissue, such as younger individuals, mammography is even less accurate. This results in a failure to detect breast cancer in many individuals, as well as incorrectly identifying benign lesions as possible cancers in many more.

In some individuals, the cancers proliferate so rapidly they result in malignancies by the time they are observed on a mammogram. Frequently, by the time one can detect a breast cancer using x-ray mammography, it may have been present in the breast for up to ten years. Mammography also cannot accurately distinguish between benign and cancerous abnormalities. Traditionally, x-ray mammography has involved solely the observation of the structural appearance of the breast tissue. Unfortunately, breast cancer often appears very similar to normal breast tissue from a structural perspective, and so the cancer goes undetected with mammography. Although use of mammography to detect breast cancer in individuals over fifty years of age may change their survival by thirty percent, there remains a need for better, more accurate and more effective methods of detection.

Scientific studies have demonstrated that x-ray mammography and ultrasound will fail to detect many, if not most, early breast cancers because they evaluate solely the appearance of the breast tissue and not at how the breast tissue functions. The methods of the present invention involve examination of breast tissue for structural abnormalities and abnormal function-not merely abnormal appearance. Because the methods of the invention evaluate both the structure and function of the breast tissue, they are able to detect breast cancer with improved degree of accuracy. In accordance with methods of the invention, several tomographic slice images through each breast are captured, whereas x-ray mammography provides only a few projectional images of all of the breast tissue collapsed upon itself. By gathering several individual tomographic images through each breast, MRI screening is not affected by breast tissue density as it does not encounter the phenomena of tissue overlap. This is particularly important for younger individuals whose breasts are typically very dense and x-ray mammography has historically been of little or no value for these individuals. This overlapping of breast tissue in x-ray mammography is one of the causes that it frequently fails to detect early breast cancers, which are curable and most important to find.

Methods of the invention are also advantageous for individuals with breast implants because it allows improved evaluation of all the breast tissue without being hindered by the presence of the implants. Breast implants can significantly obscure the surrounding breast tissue in x-ray mammography because they may overlay much of the breast tissue in the x-ray mammography images. In addition to screening for breast cancer, MRI-screening may simultaneously evaluate the implants for possible rupture or other abnormality of the breast implant.

The methods of the invention are significantly better at detecting early breast cancer than regular x-ray mammography, and thus, will detect early cancers that x-ray mammography may miss. Because the methods of the invention are more accurate than x-ray mammography, individuals who may have undergone unnecessary biopsies for benign changes of the breast detected by x-ray mammography, may not have to undergo such procedures had they been screened using the present method, which are better able to distinguish cancerous from benign abnormalities. The methods of the present invention are able to detect approximately 95% of all breast cancers about 3 mm or larger in size. The methods of the invention also permit detection of lesions located deep within the breast tissue, which are often missed during breast self-examination. The methods of the invention are also advantageous in that they do not involve exposing the breasts to potentially harmful ionizing radiation or painful compression as with standard x-ray mammography.

Medical literature has documented the superior capability of MRI in detecting breast cancer compared to the conventional methods of x-ray mammography and ultrasound. MRI has not been used in diagnostic procedures however, until after a candidate has been identified as either being at high risk or as having a suspicious mass. It has not been used as a tool for screening normal risk, asymptomatic individuals, which the present invention advantageously provides.

In comparison to MRI screening, x-ray mammography has been shown to be relatively insensitive for the detection of early cancer. Dr. Kaiser and colleagues from the University of Jena in Germany compared the ability of ten renowned radiologists from Germany and the United States to detect breast cancer using x-ray mammography and MRI in 100 individuals. (Kaiser W A, Kopans D B, Sadowsky N L, Swets J A, Getty D G, Rieser M F. ROC comparison of x-ray mammography and dynamic MR mammography in 100 consecutive cases. Radiology 1993; 189(P):137). All the radiologists were considered experts in reading x-ray mammography, but nine out of ten had no experience interpreting MRI images of breast tissue. Even without prior MRI experience, each radiologist interpreted both x-ray mammography and MRI results from each individual. With x-ray mammography only 11 breast cancers were identified. The MRI method detected 34 cancers. This study concluded that MRI has more than three times the sensitivity of x-ray mammography in the detection of breast cancer.

Even in individuals who were suspected of having recurrent breast cancer and in whom x-ray mammography was not able to make a diagnosis, MRI was shown to have dramatically improved accuracy. For example Dr. Drew and colleagues from the University of Hull in England evaluated 102 individuals with a prior history of breast cancer and who had breast conserving surgery. (Drew P J, Kerin M J, Turnbull L W. Routine screening for local recurrence following breast conserving therapy for cancer with dynamic contrast-enhanced magnetic resonance imaging of the breast. Annals of Surgical Oncology 1998; 3: 265-270). Each woman underwent a clinical examination, x-ray mammography, and an MRI. The study concluded that x-ray mammography has a sensitivity of only 67% for recurrent breast cancer whereas MRI had a sensitivity of 100%.

Multiple other scientific studies have shown MRI to have a sensitivity of detecting breast cancer between 93 to 100% with a specificity of around 70 to 90%. Similarly, multiple scientific groups have documented MRI to exclude the presence of cancer in individuals who have questionable but inconclusive x-ray mammograms.

Not only has MRI been repeatedly proven to be superior at detecting breast cancer, it has also been proven to be superior at determining the extent of cancer in the breast and whether there is more than one cancer present. In fact, multiple scientific studies have shown that MRI will alter a significant proportion of all breast surgeries because of the additional information it provides. Dr. Conrad and colleagues, from the Hjorring District Hospital in Denmark, demonstrated that it altered 51% of the surgical procedures performed for breast cancer. (Conrad C, Corfitsen M T, Glydholm N, Christiansen F L. Pre-operative MR-mammography in breast caner individuals. European Journal of Clinical Oncology 1999; 25: 142-145). They also found that x-ray mammography had missed additional cancer sites in 22% of the individuals. None of the above-mentioned studies involves MRI screening in asymptomatic, normal risk individuals.

The methods of the invention may also result in reduced healthcare costs by changing the focus of medicine from expensive and reactive cancer treatment to cost-effective, proactive early detection. The greatest cost of breast cancer screening is not the screening test performed, but the overwhelming number of unnecessary breast biopsies performed every year. Hundreds of thousands of breast biopsies are performed unnecessarily each year due to the inaccuracy of x-ray mammography and ultrasound examinations. Dr. Kopans from Harvard reports the specificity of x-ray mammography to be only 25%. (Kopans D B. Preoperative imaging-guided needle placement and localization of clinically occult lesions. In: Kopans D B, eds. Breast imaging. Philadelphia, Pa.: Lippincott, 1989; 320-341). Less than 1 out of 4 of the 750,000 breast biopsies performed each year in America turn out to be a cancer—the remainder are unnecessary, incurring only medical costs and suffering. The methods of the invention, with its dramatically improved accuracy, could prevent unnecessary biopsies, saving healthcare dollars as well as detecting breast cancer in individuals early enough that the cancer may be removed without the need for radiation, thus preventing the needless emotional and physical suffering of thousands of individuals.

Schmitt and colleagues from the University of Jena in Germany have documented the cost-effectiveness of using MRI prior to surgery, concluding that through the use of breast MRI “a major cost reduction can be achieved by prevention of unnecessary surgery, avoidance of additional surgery and associated trauma for individuals.” (Schmitt K P, Boehm T, Fleck M, Kaiser W A. Cost-effectiveness of MR-imaging in the preoperative work-up of suspicious breast lesions. Rofo Fortschr Geb Rontgenstr Neuen Bildgeb Verfahr 2001; 173(10): 898-901).

In those individuals found to have breast cancer, the cost of treatment differs dramatically in those detected early compared to those found late. While the average cost of early breast cancer treatment typically ranges from $10,000 to $50,000, treatment of late stage breast cancer can range between $80,000 and $200,000. Because the methods of the invention can detect early breast cancer with much higher accuracy, individuals can be treated early when the costs are a fraction of what they would be otherwise. Most importantly, according to the American Cancer Society, when breast cancer is detected early, over 96% of all individuals will survive. If found late, less than 1 out of 4 will survive. (Breast Cancer Facts and Figures 2001-2002, American Cancer Society).

The diagnostic benefits arising from the present invention include the overall management of breast cancer in individuals. The diagnosis of breast cancer at an earlier stage allows an individual more choice in the selection of various treatment options and a much higher probability of survival.

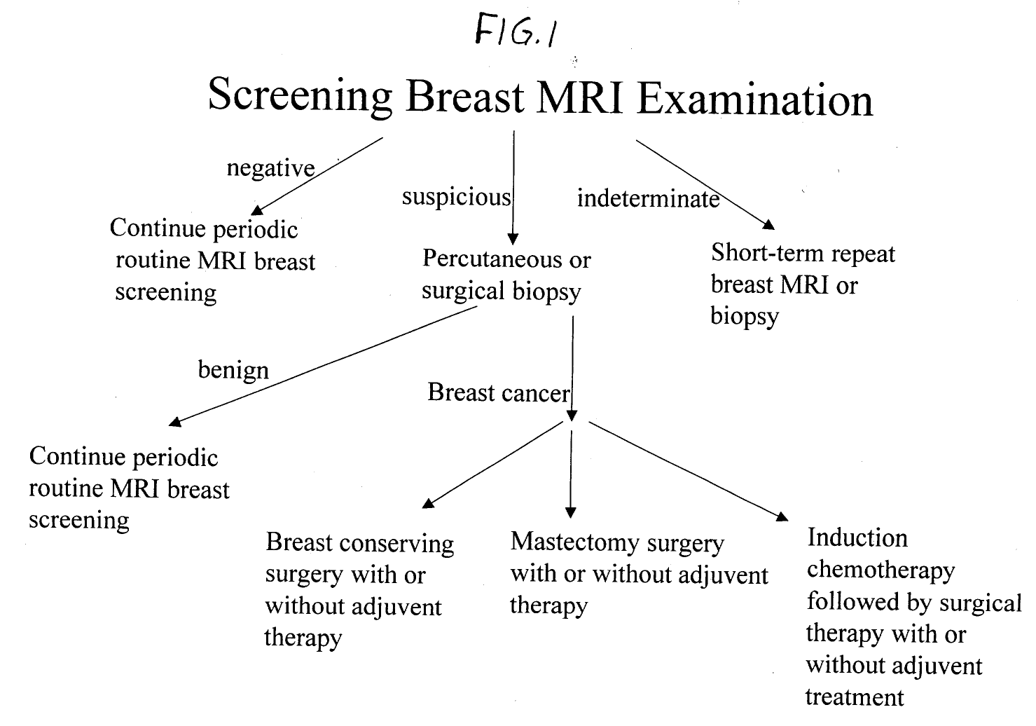

FIG. 1 is a flow chart representative of an algorithm showing assessment of diagnostic information obtained using MRI to screen normal risk, asymptomatic individuals for breast cancer. As depicted in FIG. 1, individuals are first screened for breast cancer using MRI in accordance with the present methods. If the screening is negative, i.e., shows no indications of breast cancer, the individual should continue periodic (e.g., yearly depending on age) routine MRI breast screening. If the screening results in some indication of suspicious tissue or physiological activity, then a percutaneous or surgical biopsy should be performed. If the biopsy indicates that the suspicious tissue or lump(s) is benign, the individual should continue periodic (e.g., yearly depending on age) routine MRI breast screening. If the biopsy indicates that the suspicious tissue or lump(s) is breast cancer, then depending on the size of the lump, spread of the cancer, and other factors, the physician may recommend one or more of several treatment options including, for example, (1) breast conserving surgery with or without adjuvent therapy; (2) mastectomy surgery with or without adjuvent therapy; and (3) induction chemotherapy followed by surgical therapy with or without adjuvent treatment. Adjuvent therapy or treatment means supplementary treatment such as performing radiation therapy or chemotherapy in addition to surgical intervention. If the screening is indeterminate, i.e., it is not suspicious, but it cannot be said to show no indications of breast cancer, the individual should repeat the breast MRI within a short period of time (e.g., within 3 to 6 months of the first MRI) or have a percutaneous or surgical biopsy performed as described above.

The following examples illustrate specific embodiments of the invention. The examples set forth herein are meant to be illustrative and should not in any way serve to limit the scope of the claimed invention. As would be apparent to skilled artisans, various changes and modifications are possible and are contemplated within the scope of the invention described.

### EXAMPLES

### Example 1

MRI is performed on the breasts of a normal risk, asymptomatic individual. Several images of the breasts are acquired using various MRI sequences to produce T1, T2 and/or Proton Weighted images of the breasts. The images are then reviewed by a board-certified radiologist utilizing specialized medical workstations, which detect any indications of breast cancer.

### Example 2

A dynamic investigation is performed on an individual, in which the enhancement behavior of a contrast agent is evaluated. A baseline pre-contrast MRI of the entire breast is performed with a T1 weighted image sequence. A gadolinium contrast agent is then injected into a vein in the arm of a individual in a dose of approximately 15 mL. The individual is then injected with a physiological saline solution of about the same amount (i.e., approximately 15 mL), so that the contrast agent reaches the blood circulation as completely as possible.

By administering intravenous gadolinium in a dosage around 0.1 mmol/kg of body weight and performing a dynamically enhanced MRI with T1 weighting of the breast over a period of minutes at specific intervals, cancer from normal breast tissue can be differentiated with a very high degree of sensitivity and specificity.

The gadolinium contrast agent is administered at a bolus rate of approximately 2 cc/sec and subsequent MRI imaging through the breast is then acquired at 1 minute intervals. In so doing the degree of percentage of MRI signal enhancement over time is plotted. Regions of cancer growth within the breast demonstrate a significantly higher degree of enhancement than surrounding normal breast tissue and this degree of enhancement tends to decrease more quickly than that of the normal tissue. A board certified radiologist identifies any regions of possible cancer from the normal breast tissue based on the degree of enhancement in addition to the structural characteristics of the tissues present in the MRI images.

### Example 3

Dynamically enhanced MRI is conducted as set forth in Example 2, however the contrast agent examinations are conducted bilaterally, i.e., both breasts are examined simultaneously. The MRI is then performed either in the transversal, coronal or other suitable slice orientation that allows imaging of both breasts simultaneously.

### Example 4

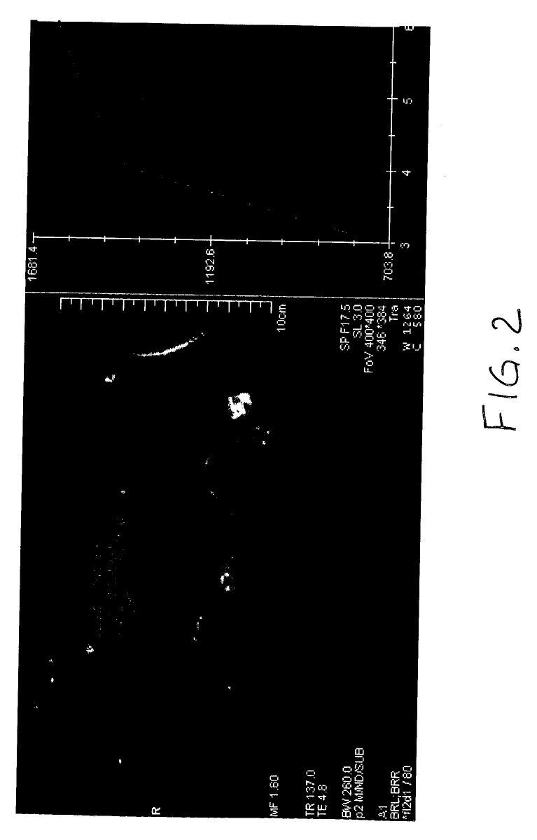

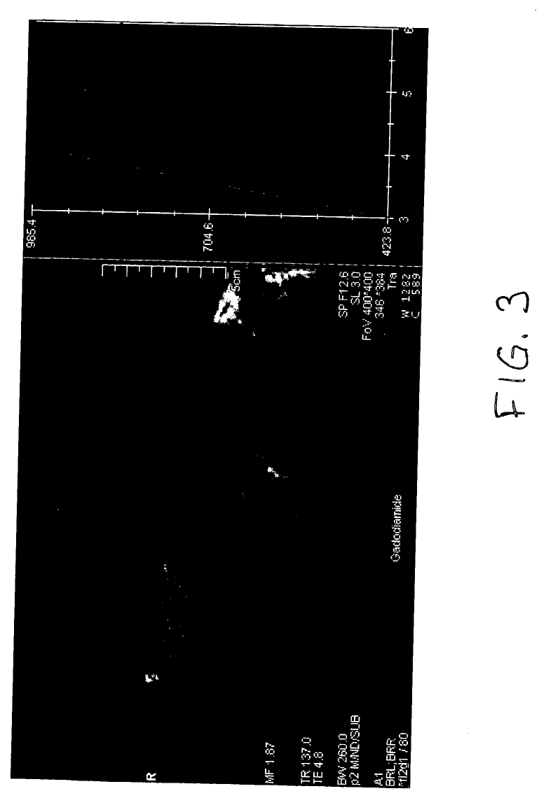

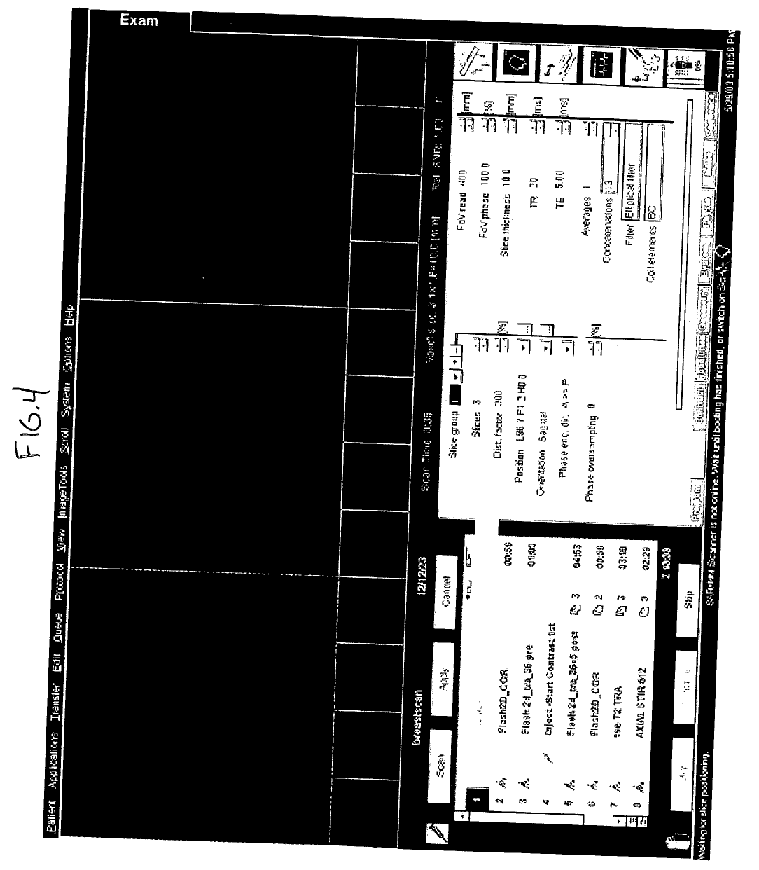

The breasts of individuals were examined using MRI in accordance with the methods of the present invention. FIGS. 2-3 are sample curves demonstrating relative enhancement over time for a benign nodule in the breast (FIG. 2) and for a cancer (FIG. 3). The cancer in FIG. 3 shows dramatic early enhancement (of at least 90% more than in the pre-contrast image) and tends to wash out after the first or second minute (meaning the amount of enhancement decreases quickly). A benign region of enhancement as depicted in FIG. 2 generally shows steadily increasing enhancement over time and usually will demonstrate less than 90% enhancement in the first minute after injection.

### Example 5

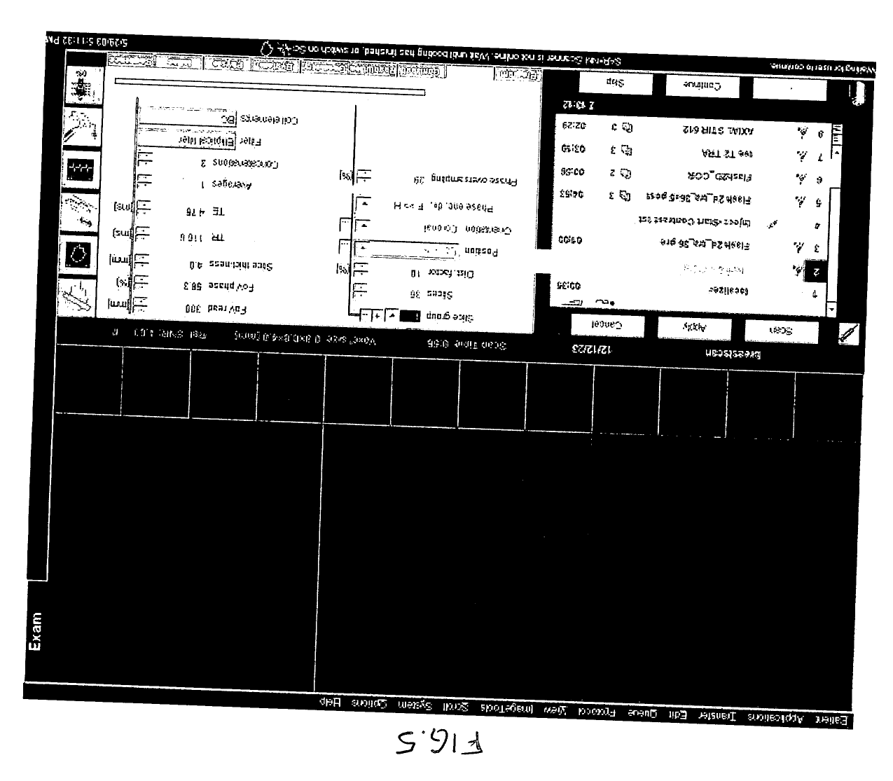

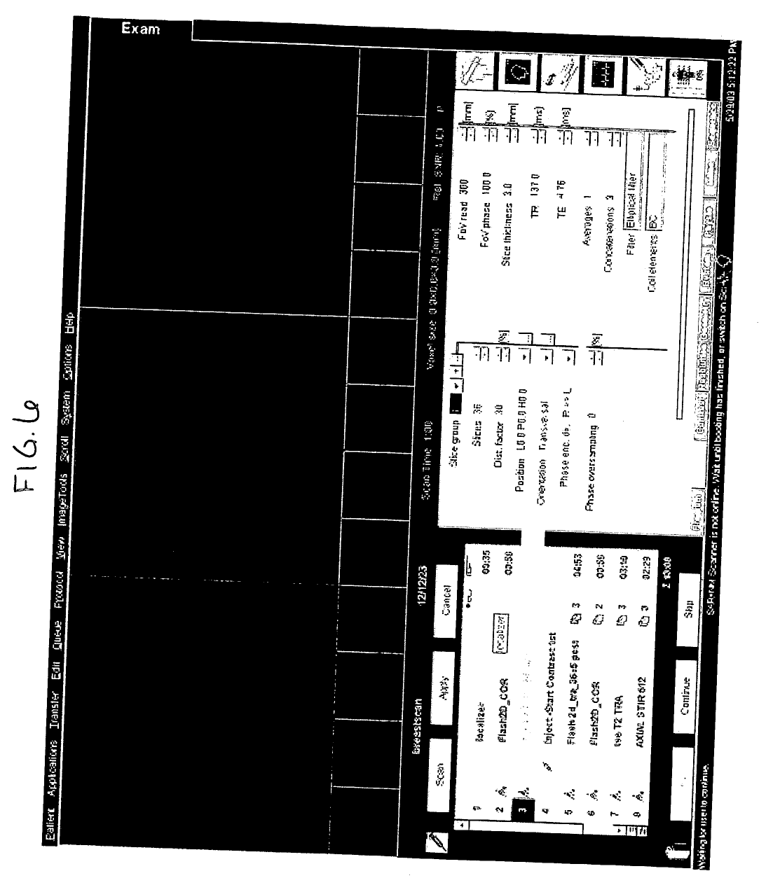

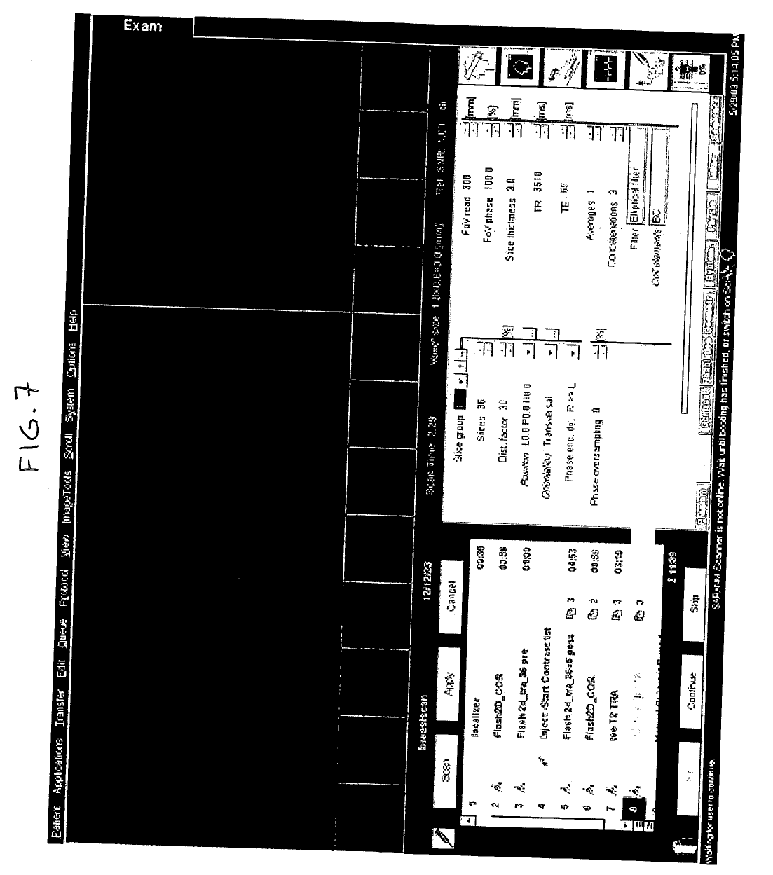

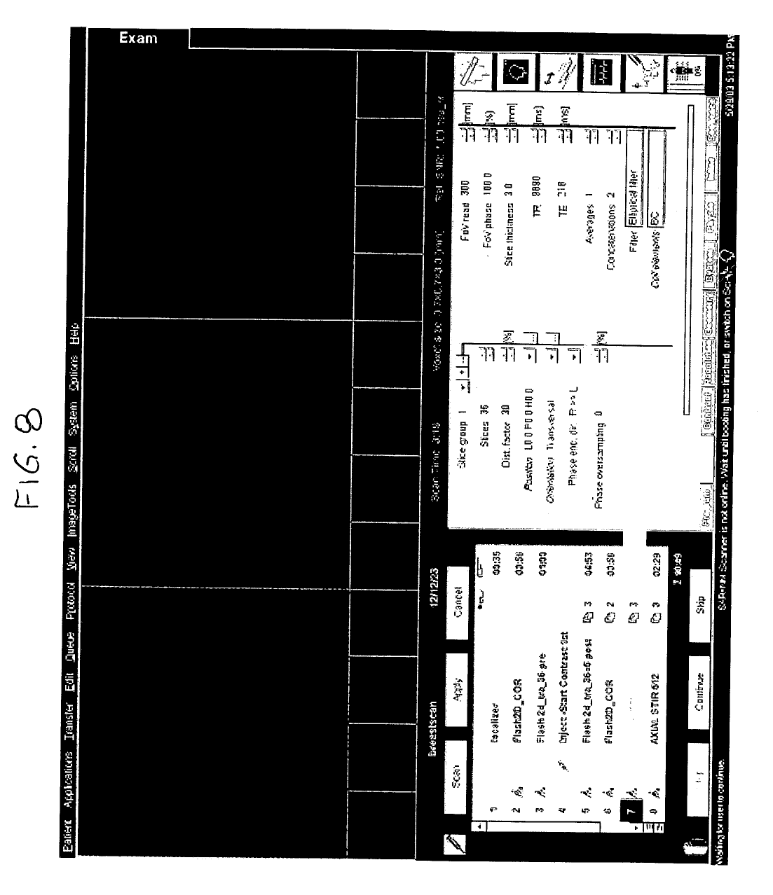

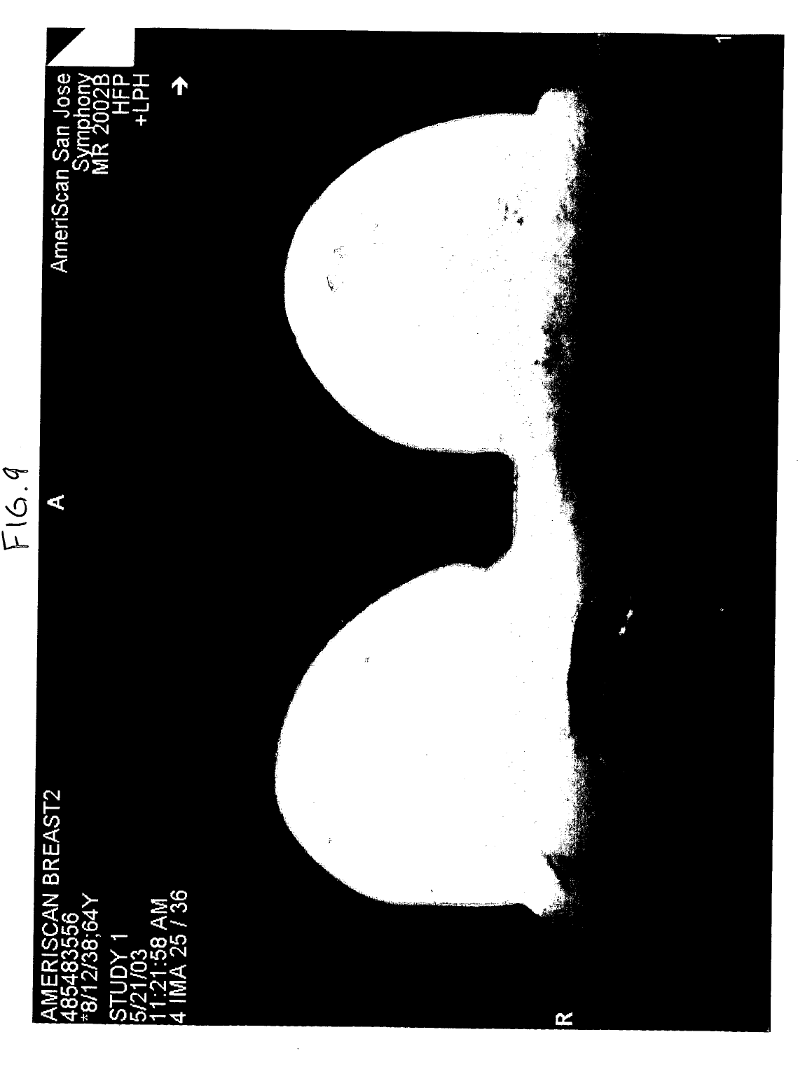

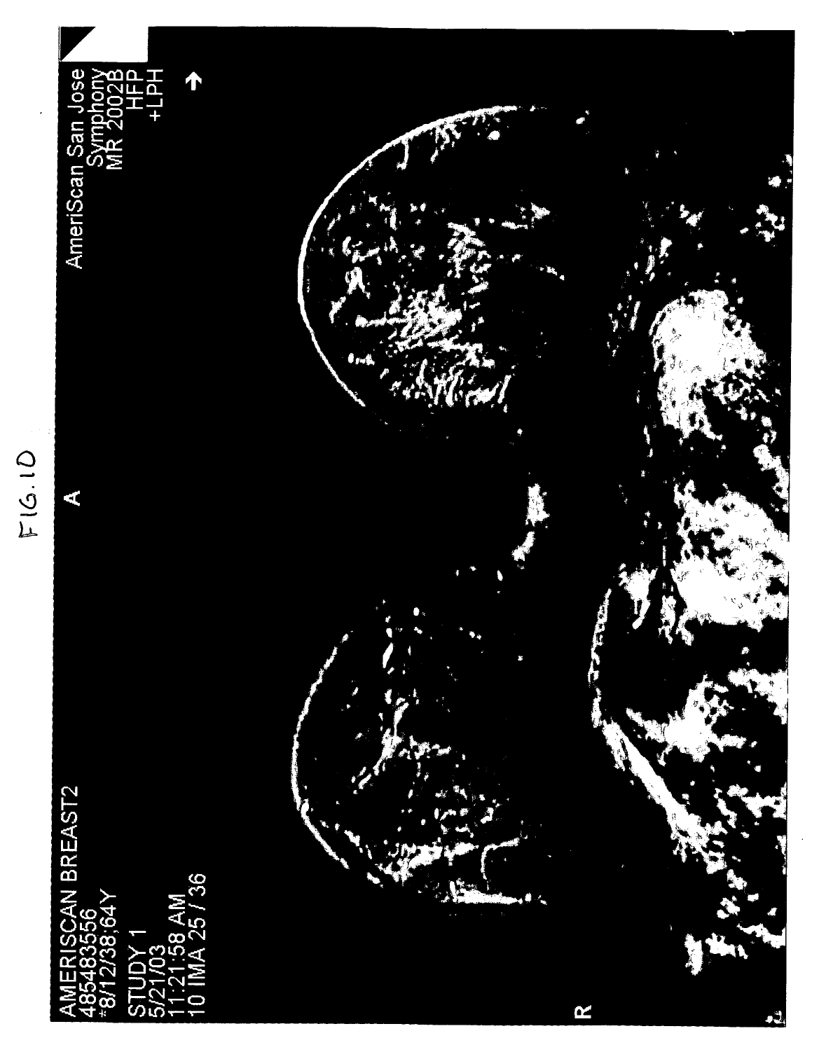

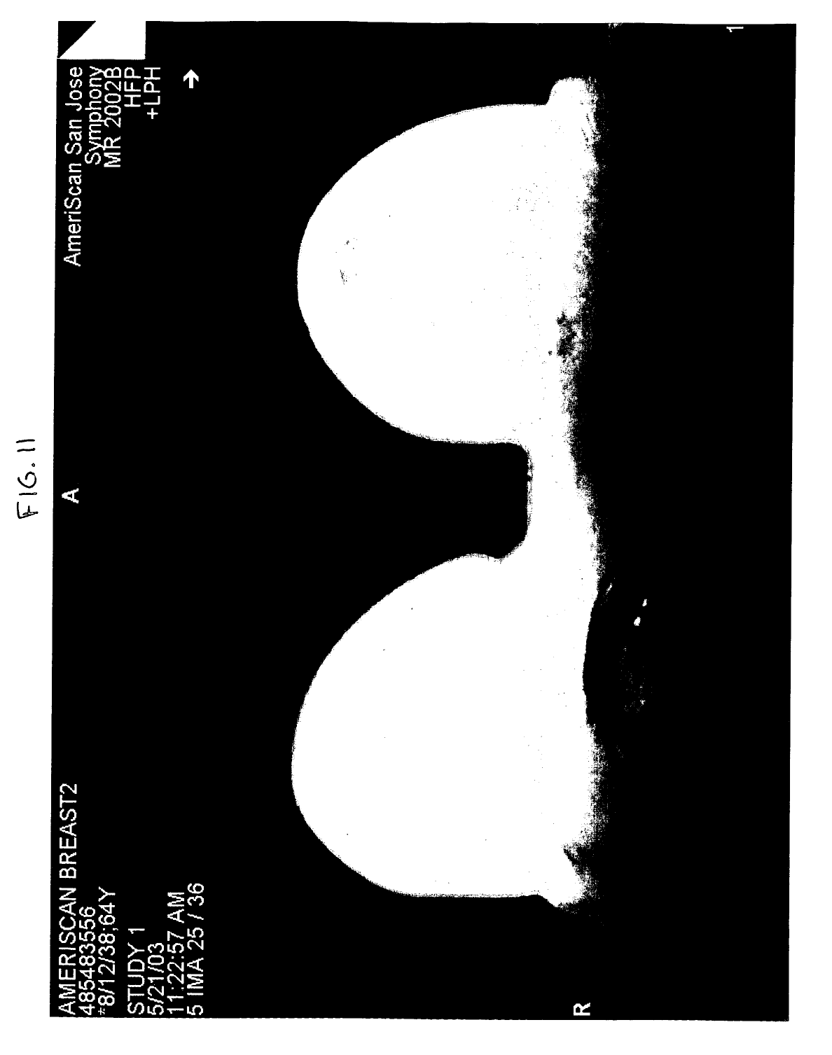

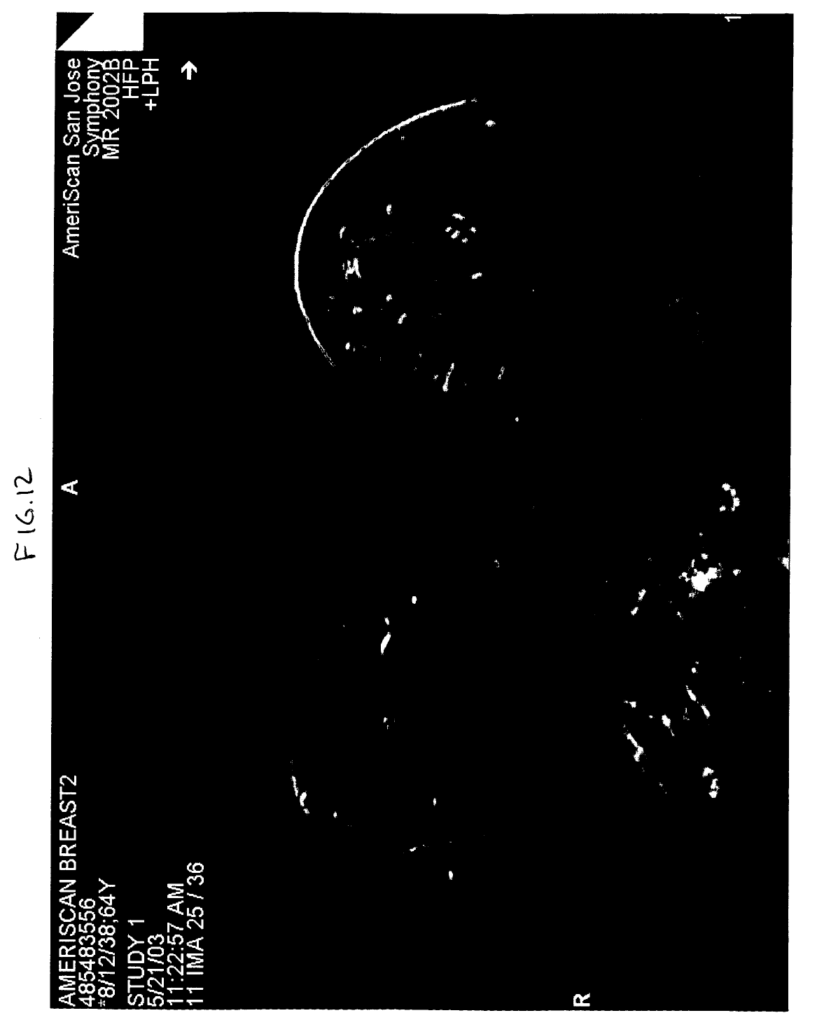

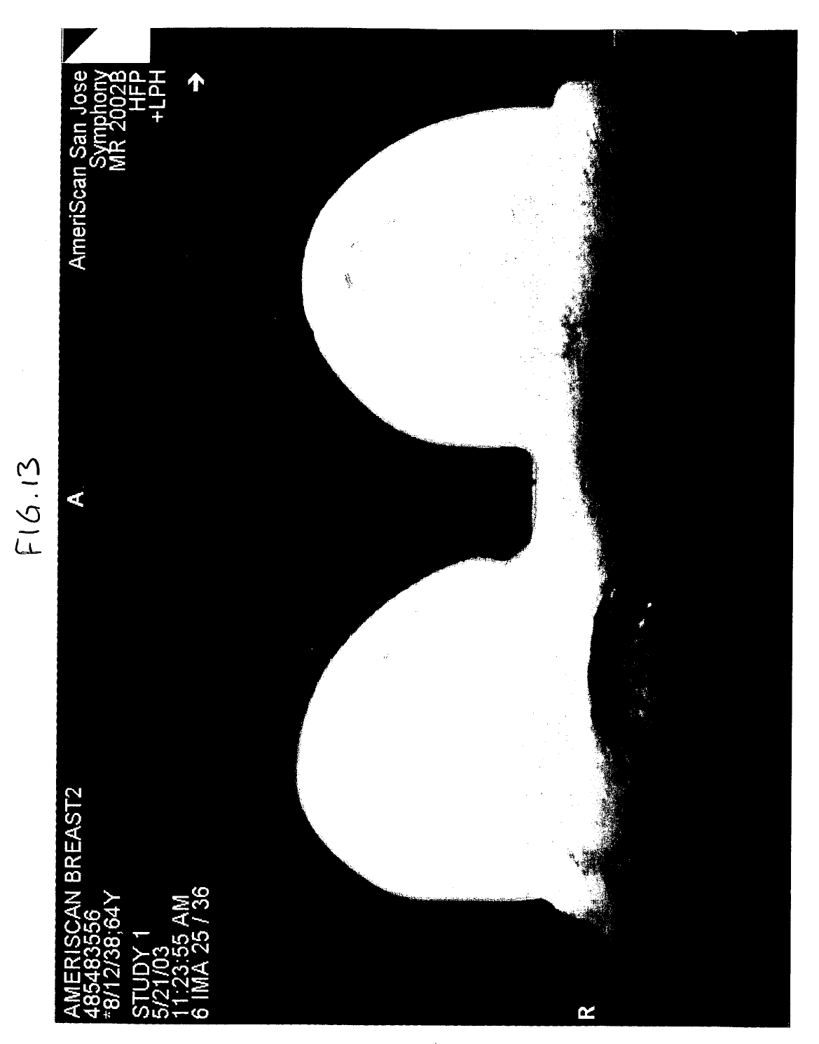

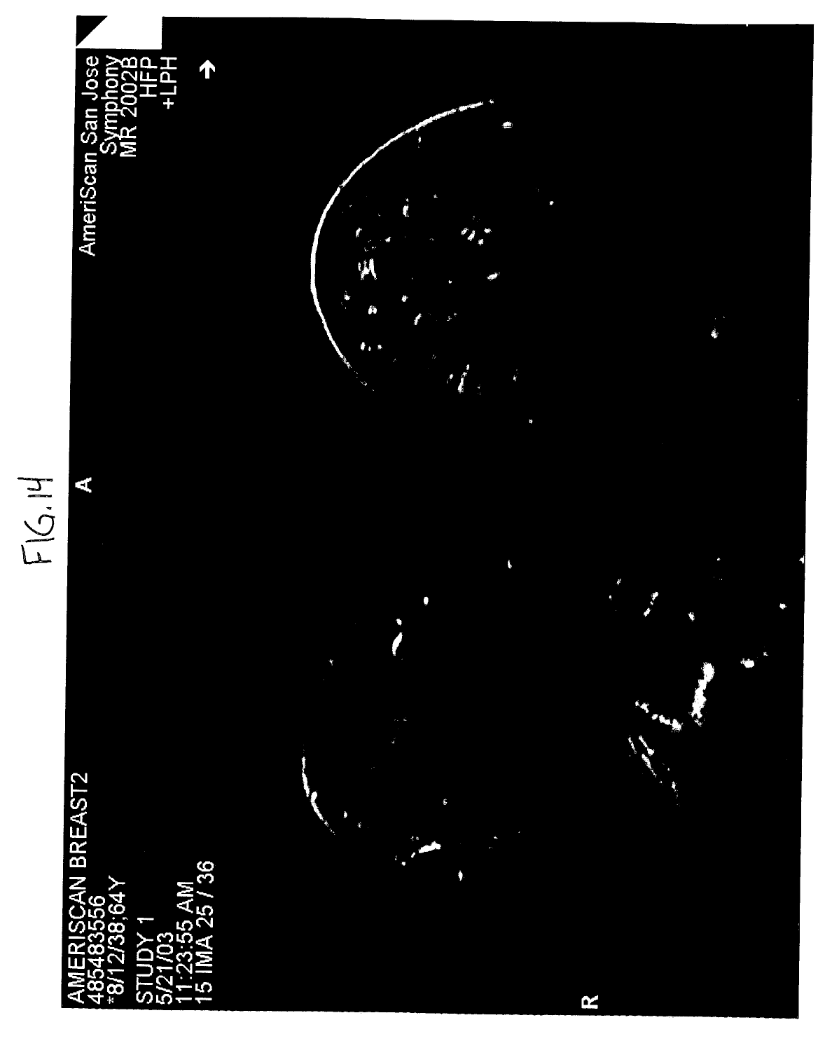

The control panel of an MRI may be set up as depicted in FIGS. 4-8, for imaging protocols in accordance with methods of the invention. In the bottom right corner is the ‘task card’ that demonstrates all of the various settings such as the TE, TR, etc. The localizing protocol (FIG. 4) is run before any of the other protocols to get the breasts lined up in the field of view of the images. The coronal imaging protocol (FIG. 5) is looking at the breasts from a ‘front on’ view. The other protocols, including a pre-contrast and post-contrast protocol (FIG. 6), a STIR imaging protocol (FIG. 7), and a T2 imaging protocol (FIG. 7), are done from an ‘axial’ or ‘transverse’ view. On the bottom left of the images in FIGS. 4-8 is the over protocol sequence (numbered 1 through 8) which shows the order of each sequence to be run. As would be apparent to one skilled in the art, these figures are demonstrative only, and the MRI control panel may be modified at various stages as desired or required, in accordance with the present methods.

### Example 6

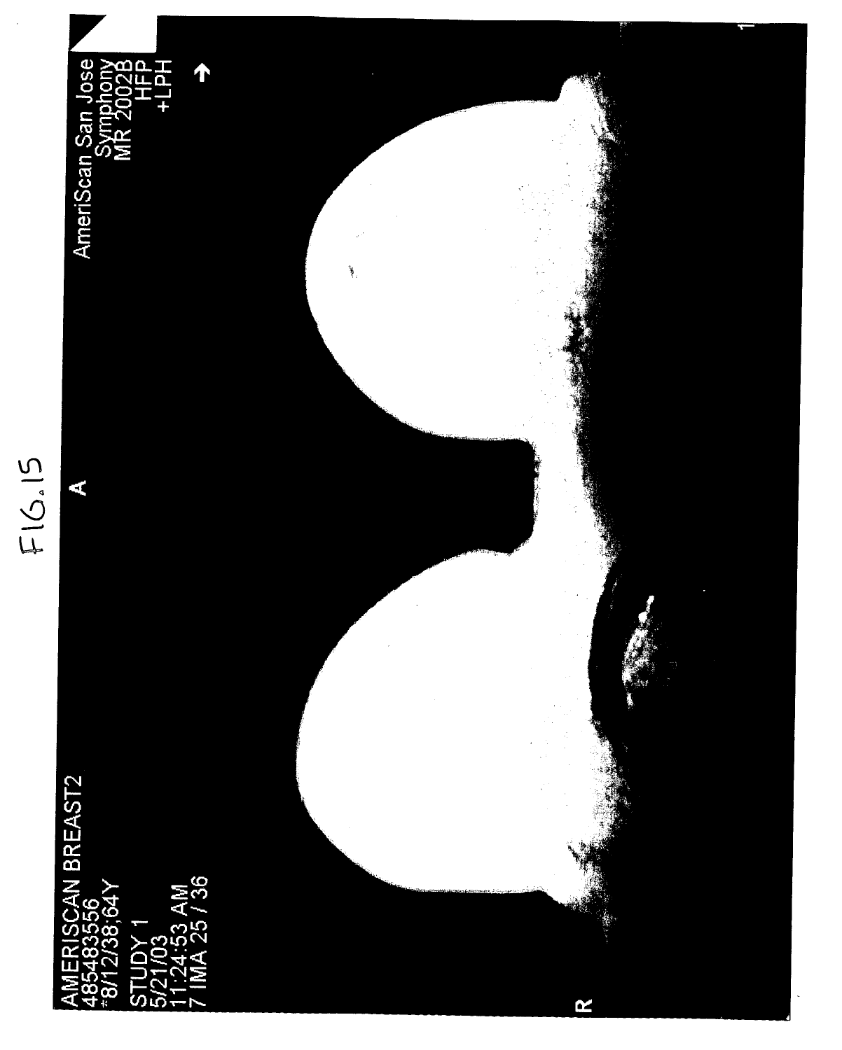

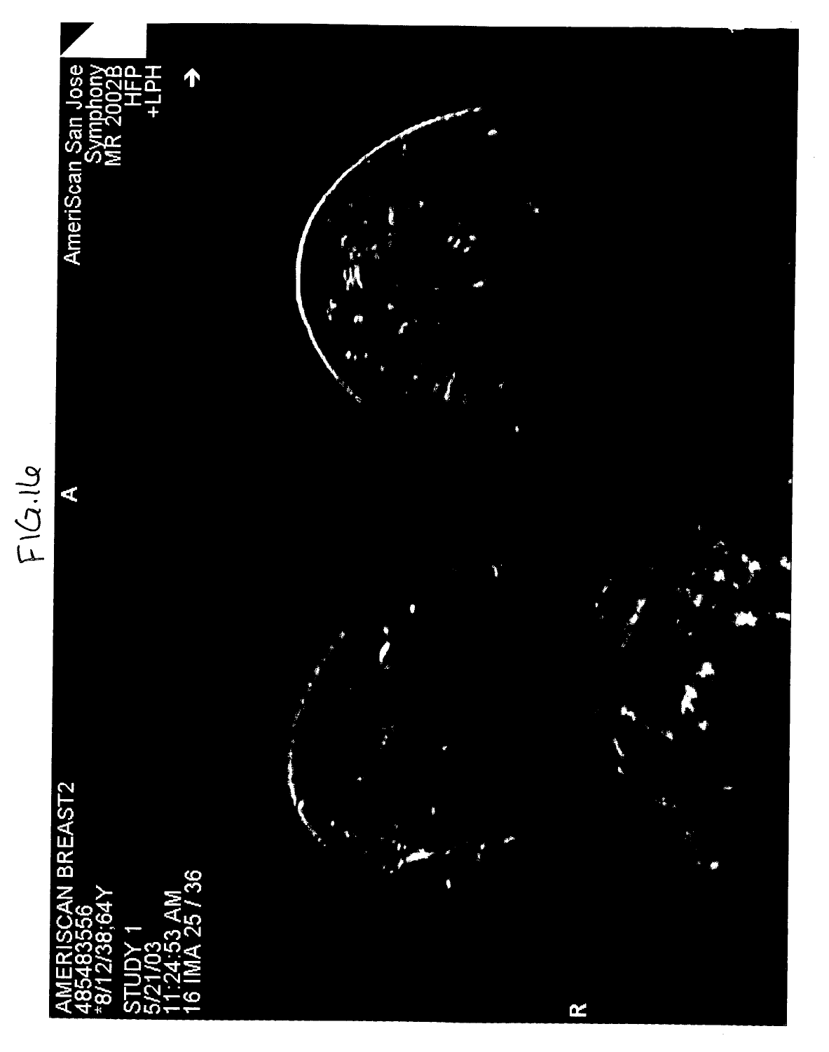

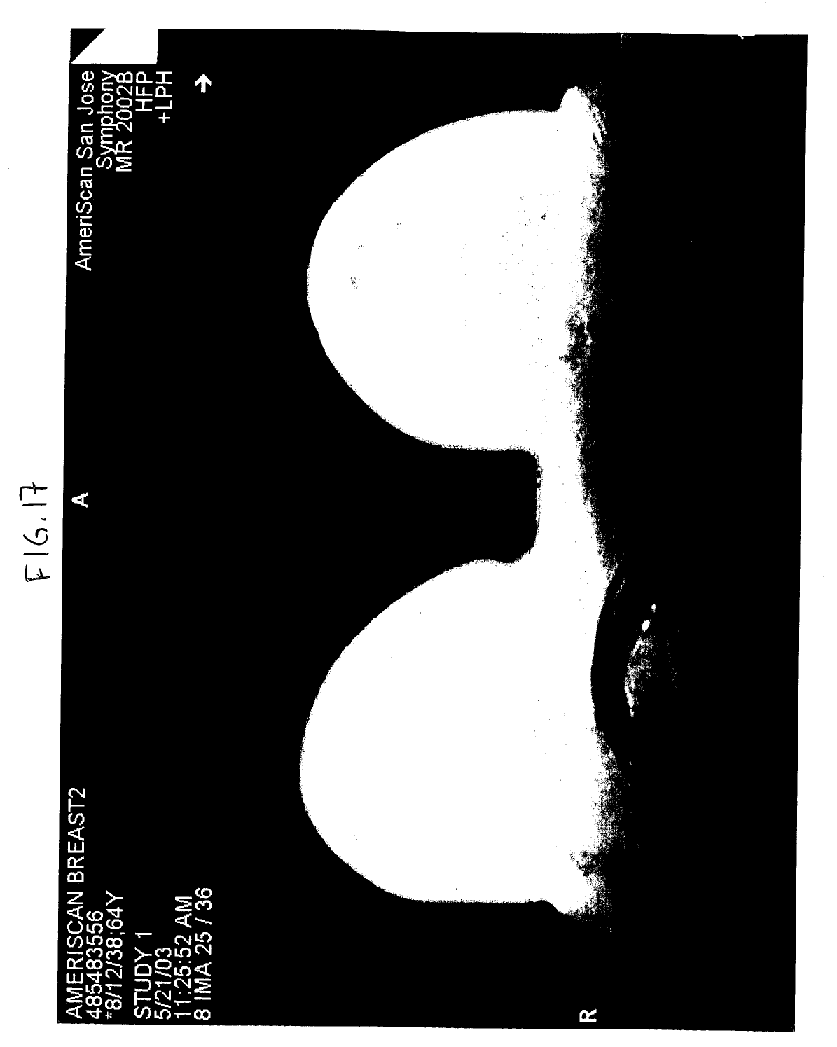

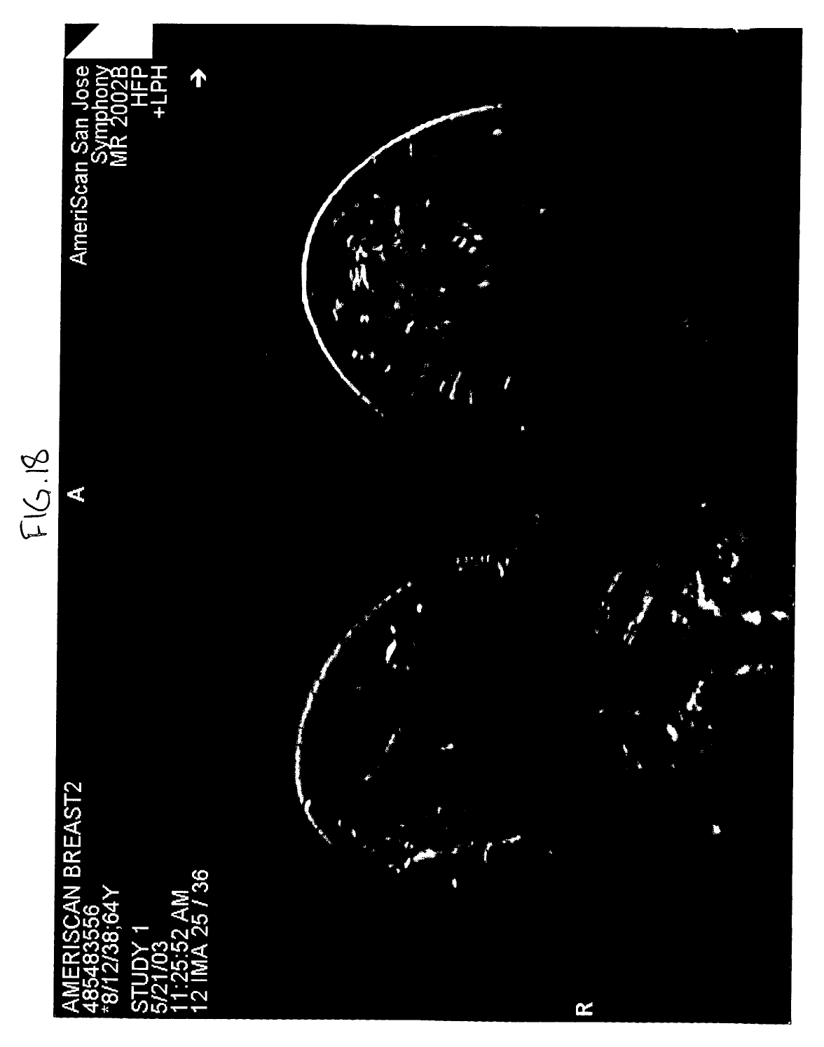

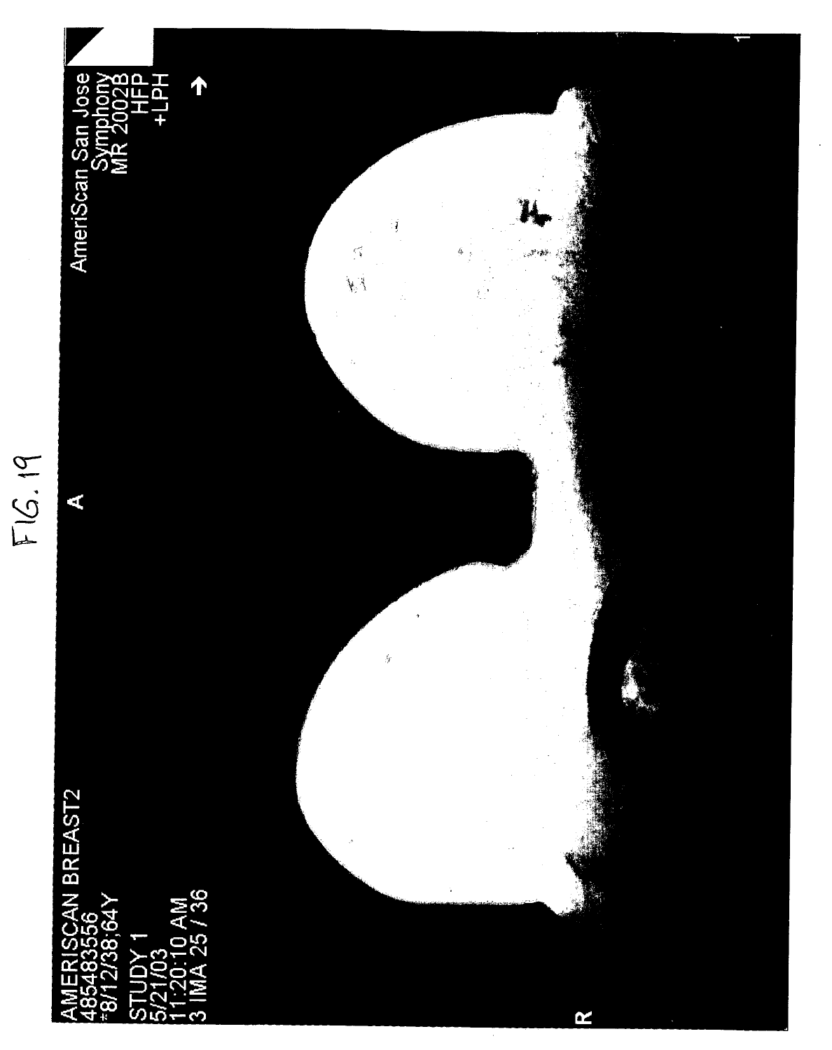

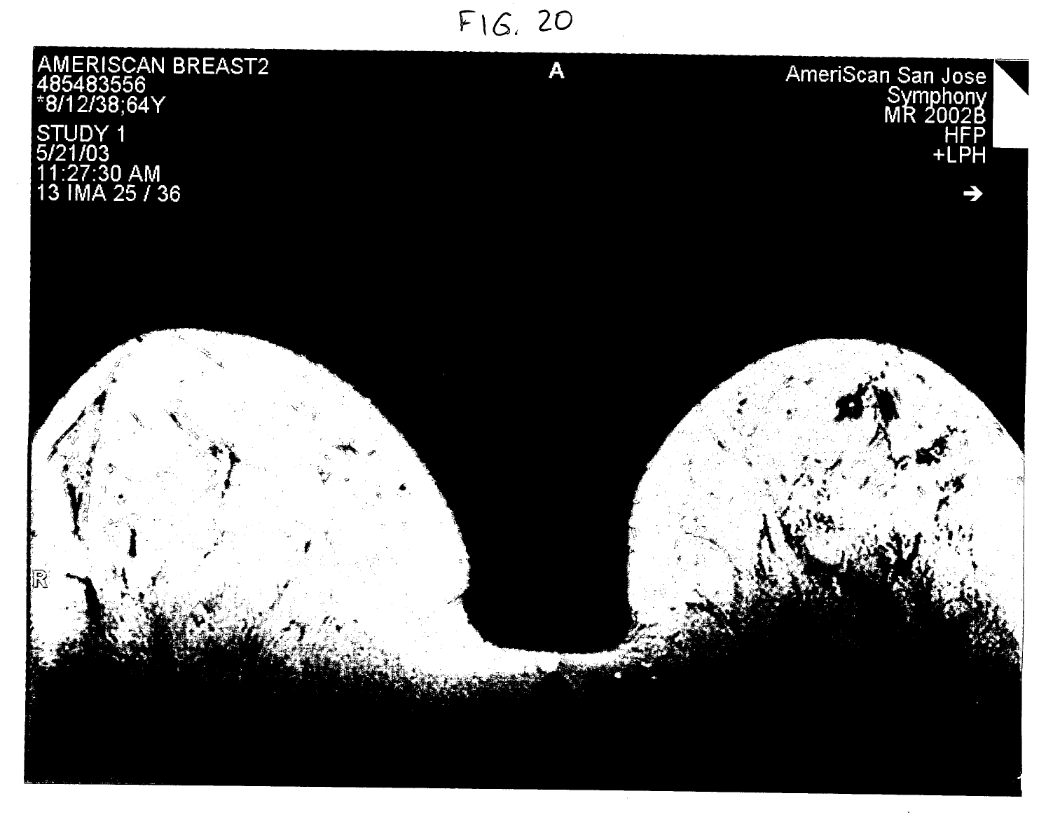

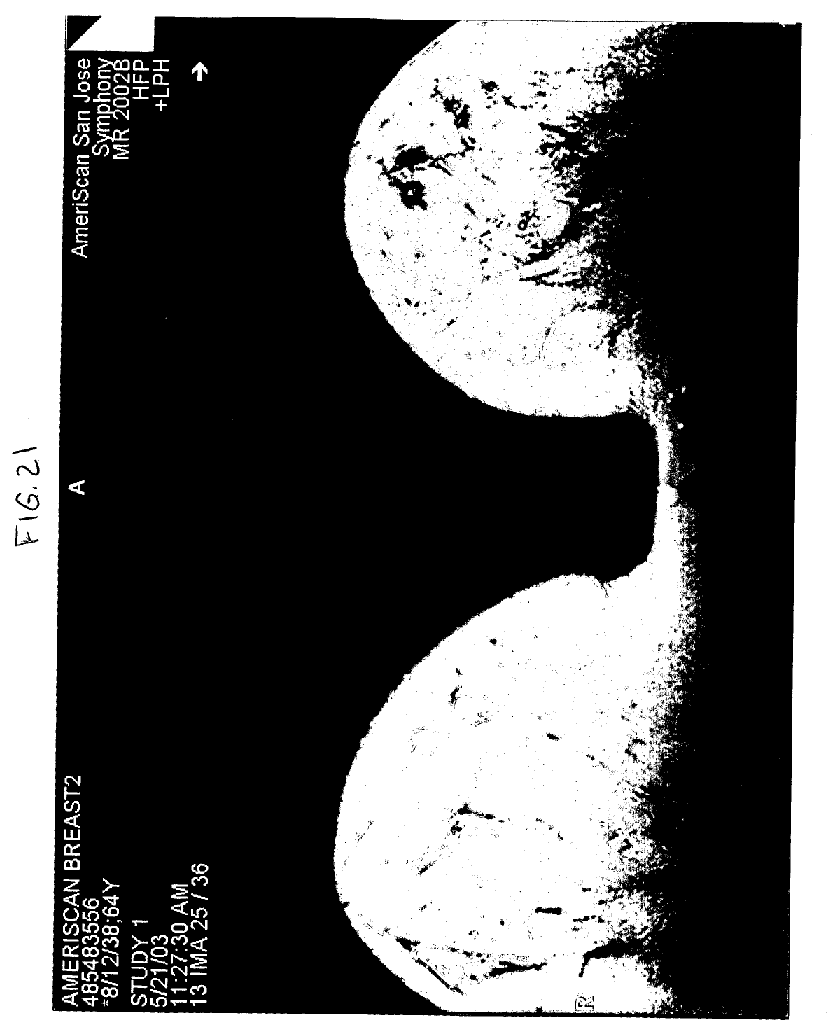

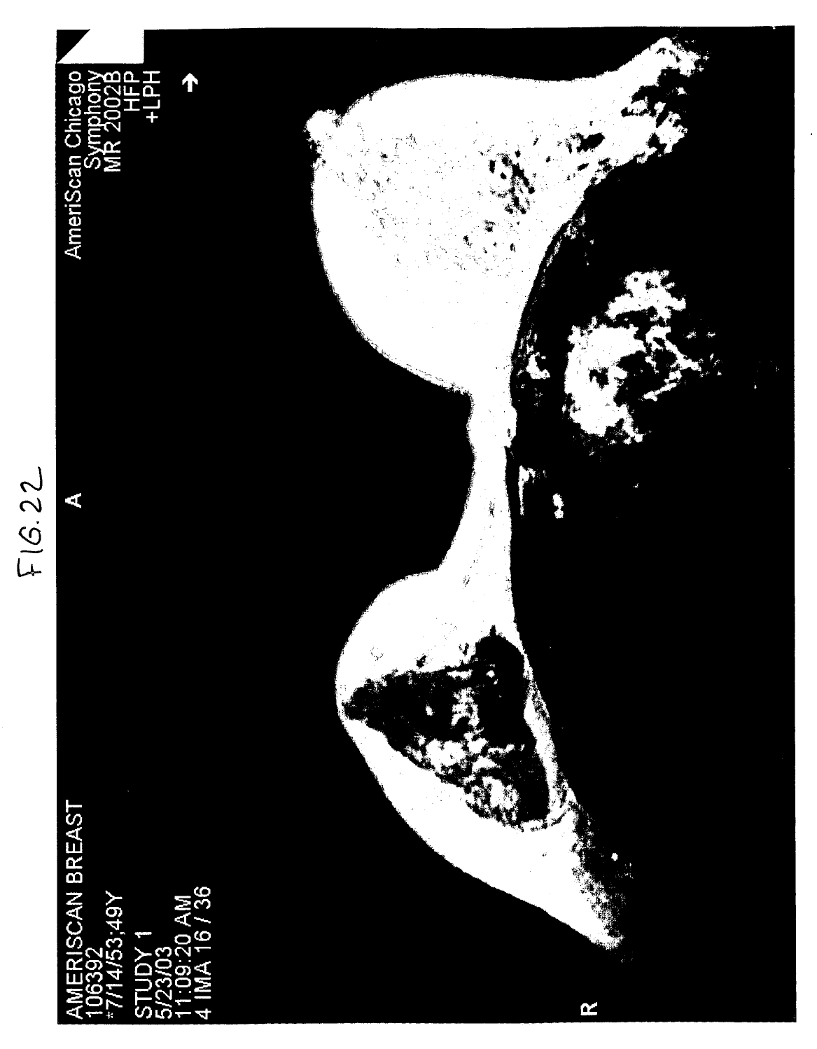

The breasts of an individual were examined using MRI in accordance with the methods of the present invention. FIGS. 9-21 depict sample MRI images of an individual who had a normal examination according to the methods of the present invention. FIG. 9 is a 1 minute post contrast image. FIG. 10 is a 1 minute subtraction image wherein the image data from the pre contrast image was subtracted from that obtained in the 1 minute post contrast image. FIG. 11 is a 2 minute post contrast image. FIG. 12 is a 2 minute subtraction image wherein the image data from the pre contrast image was subtracted from that obtained in the 2 minute post contrast image. FIG. 13 is a 3 minute post contrast image. FIG. 14 is a 3 minute subtraction image wherein the image data from the pre contrast image was subtracted from that obtained in the 3 minute post contrast image. FIG. 15 is a 4 minute post contrast image. FIG. 16 is a 4 minute subtraction image wherein the image data from the pre contrast image was subtracted from that obtained in the 4 minute post contrast image. FIG. 17 is a 5 minute post contrast image. FIG. 18 is a 5 minute subtraction image wherein the image data from the pre contrast image was subtracted from that obtained in the 5 minute post contrast image. FIG. 19 is a pre contrast image. FIG. 20 is a STIR image. FIG. 21 is a T2 weighted image. A trained radiologist or other person determined from these images that the scanned individual did not have indications of breast cancer.

### Example 7

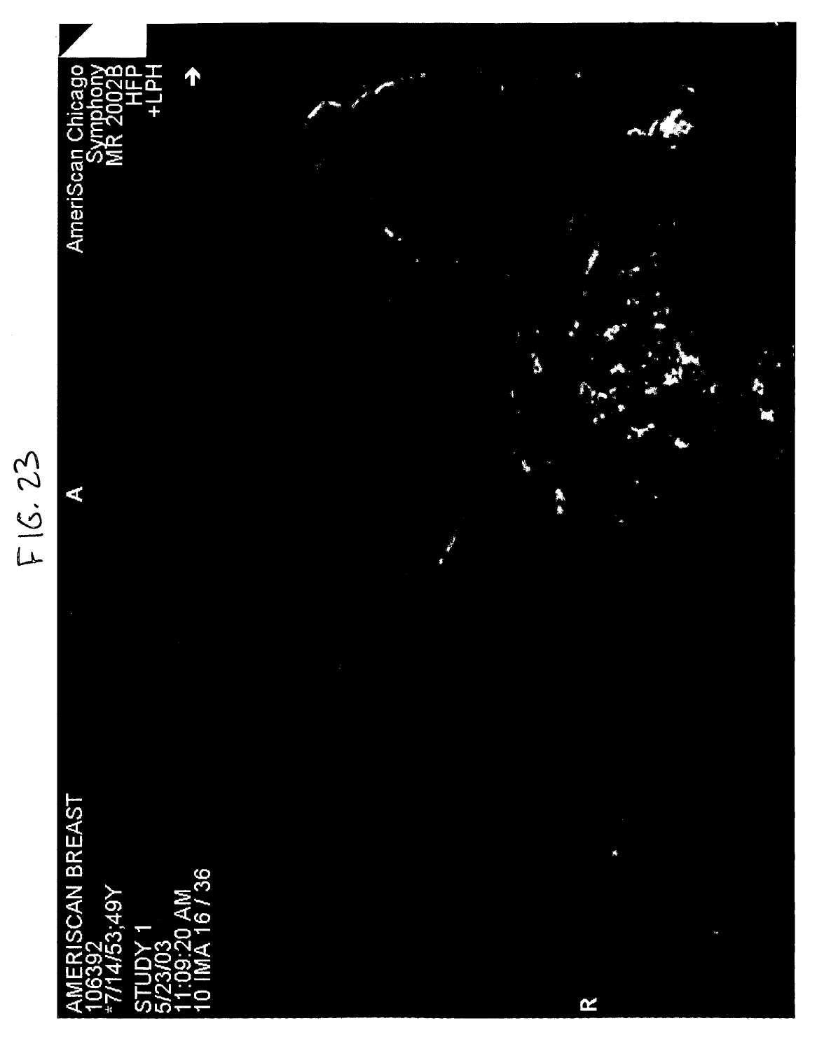

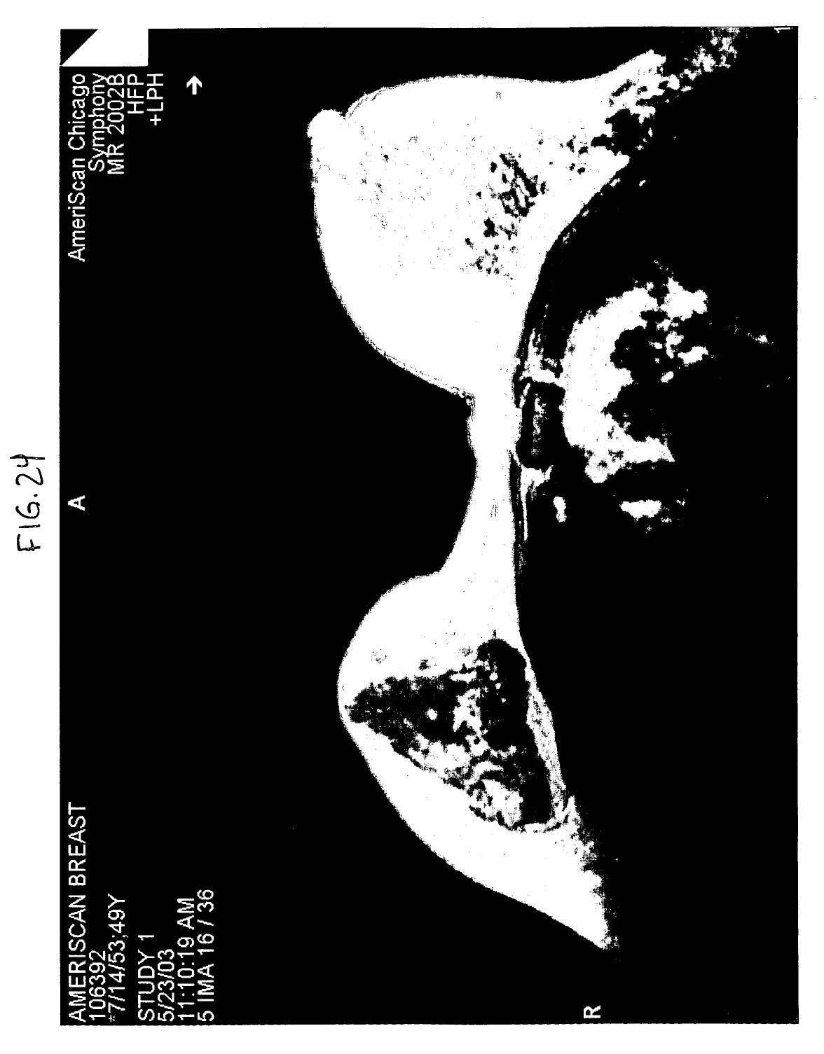

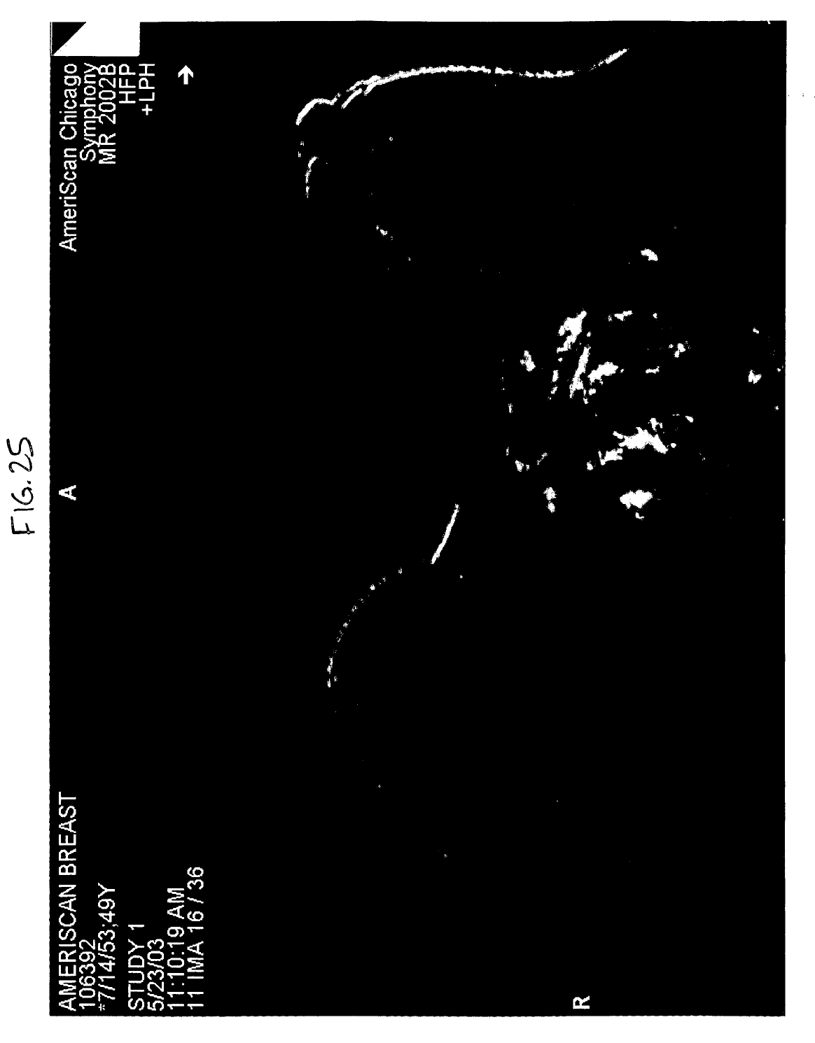

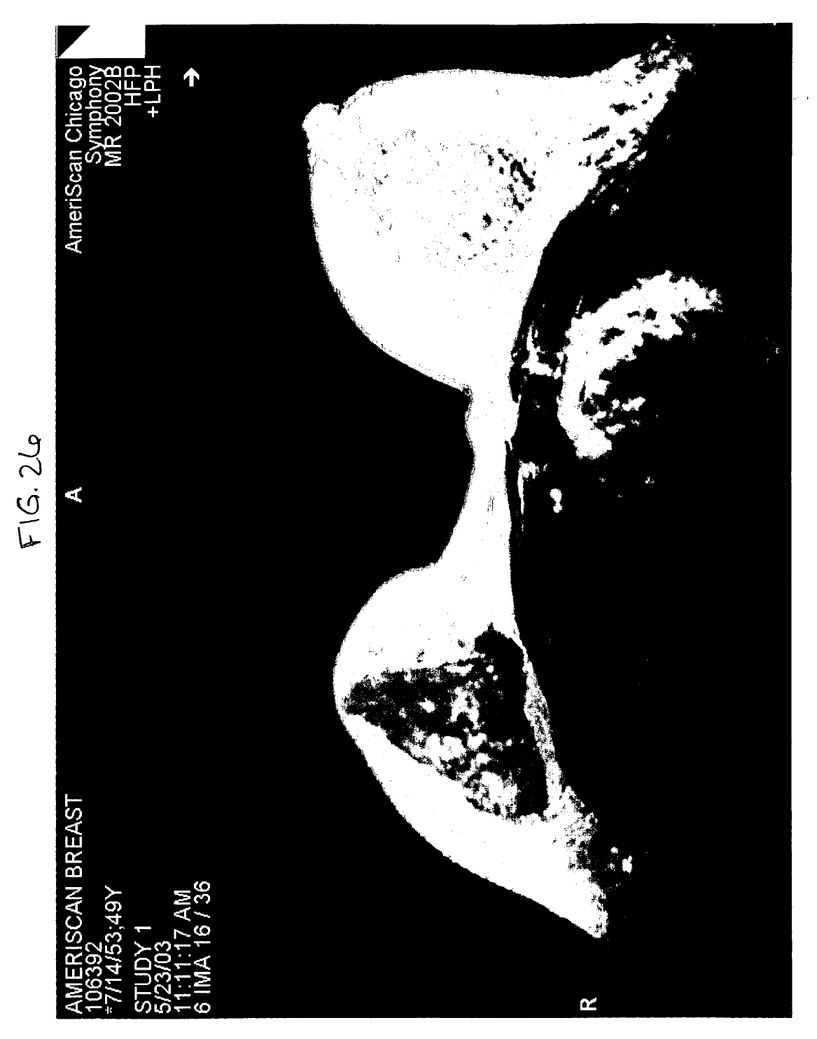

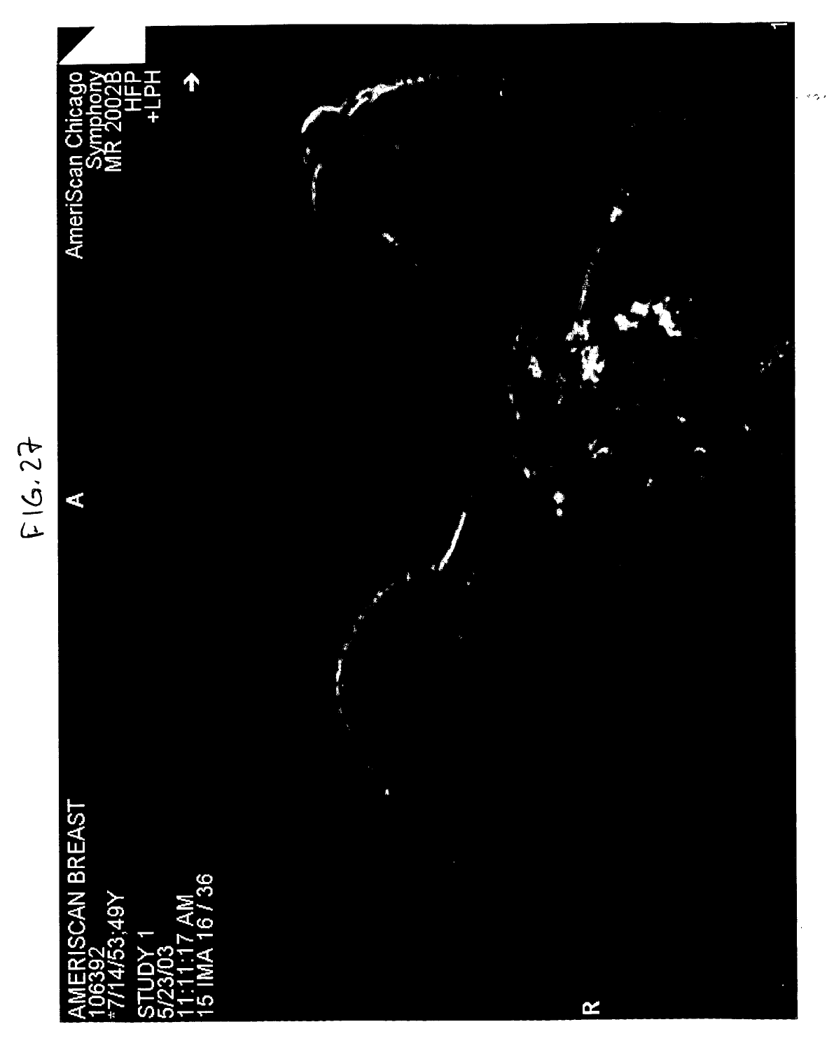

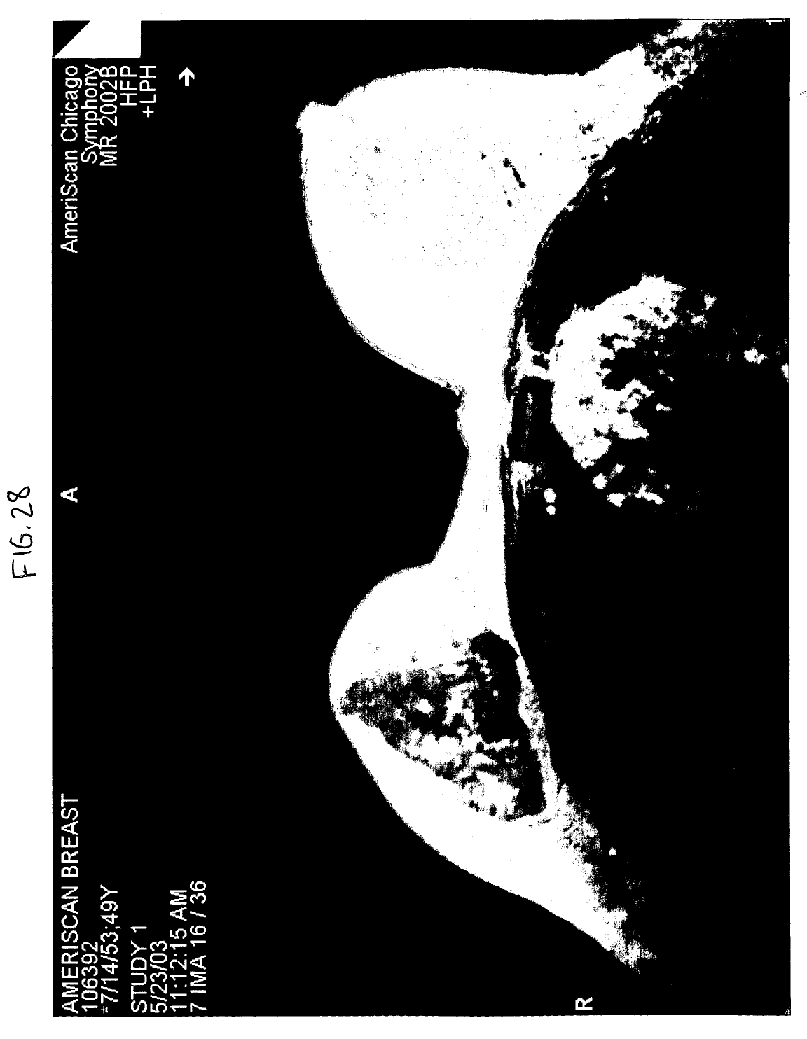

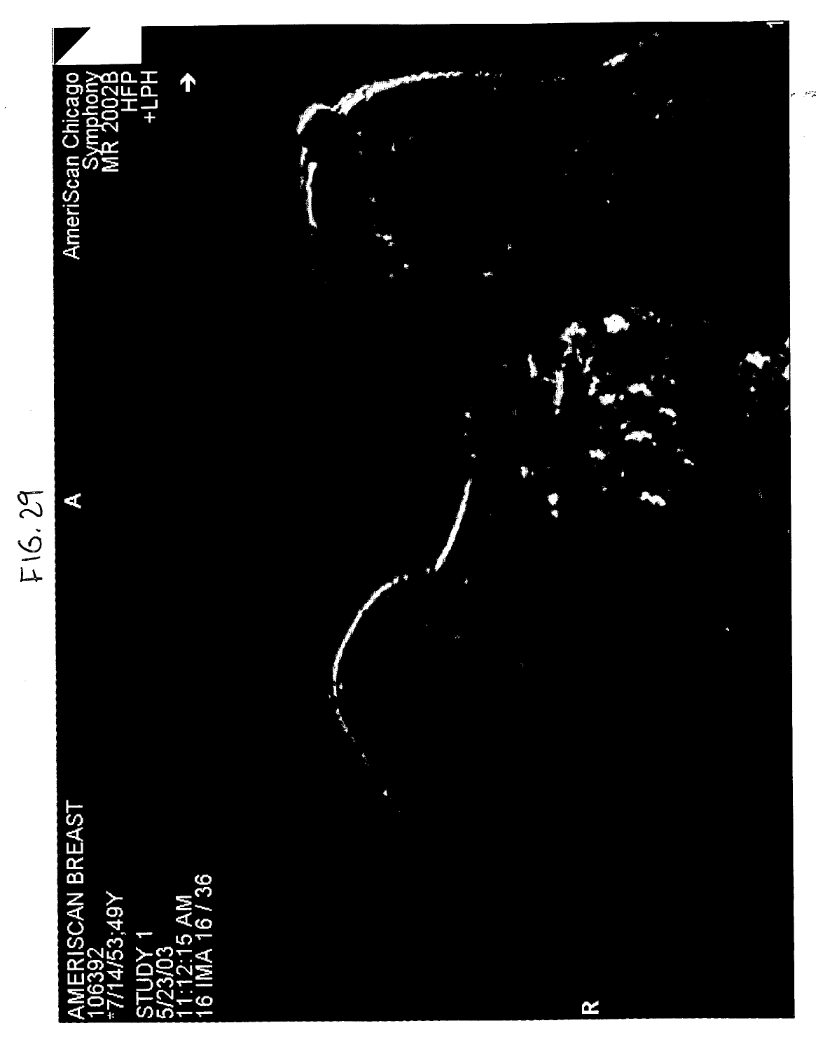

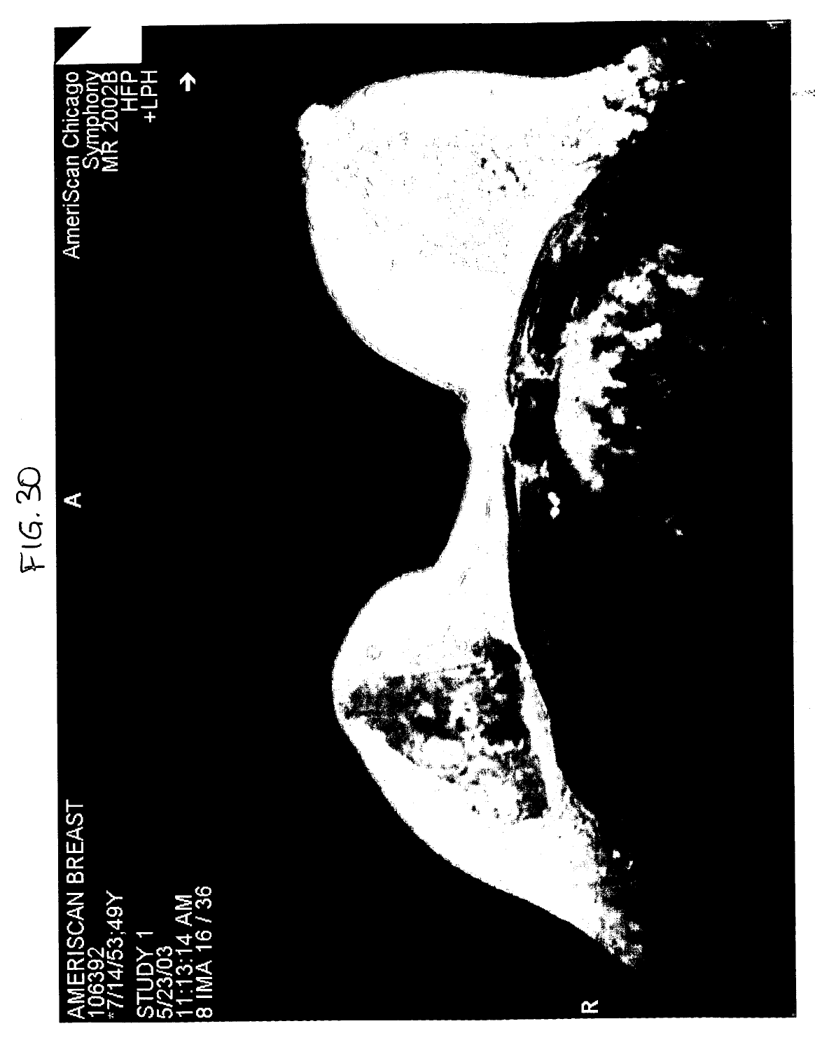

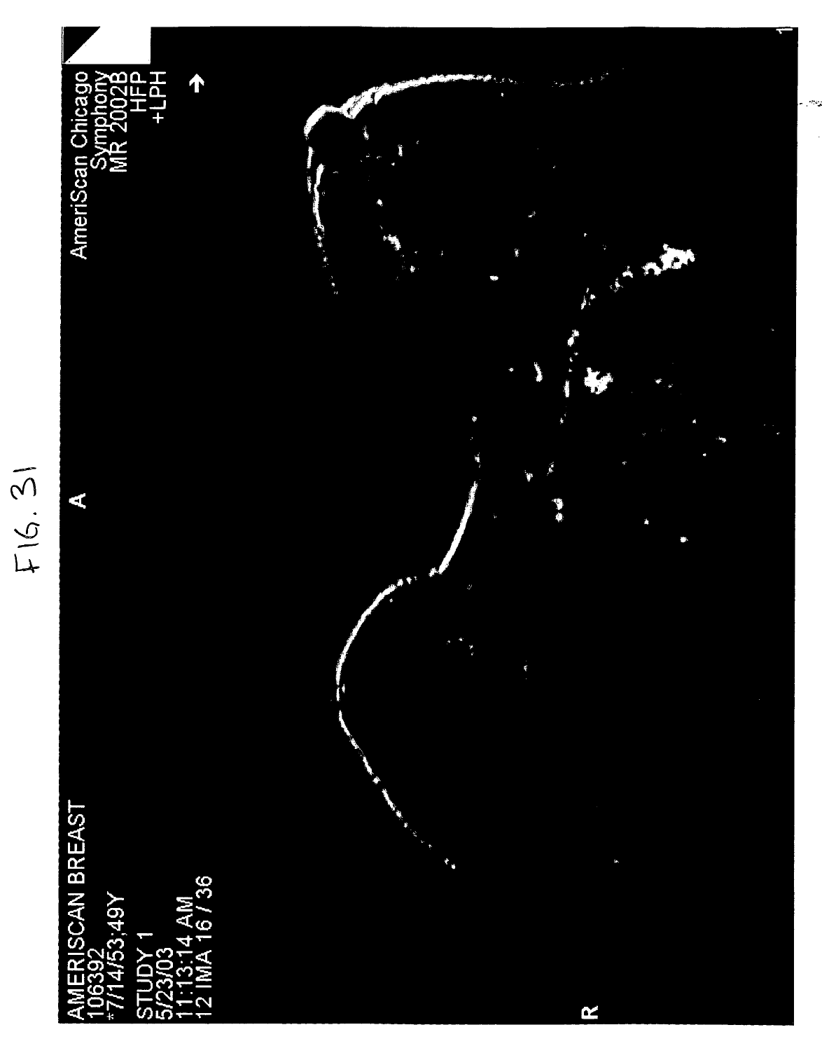

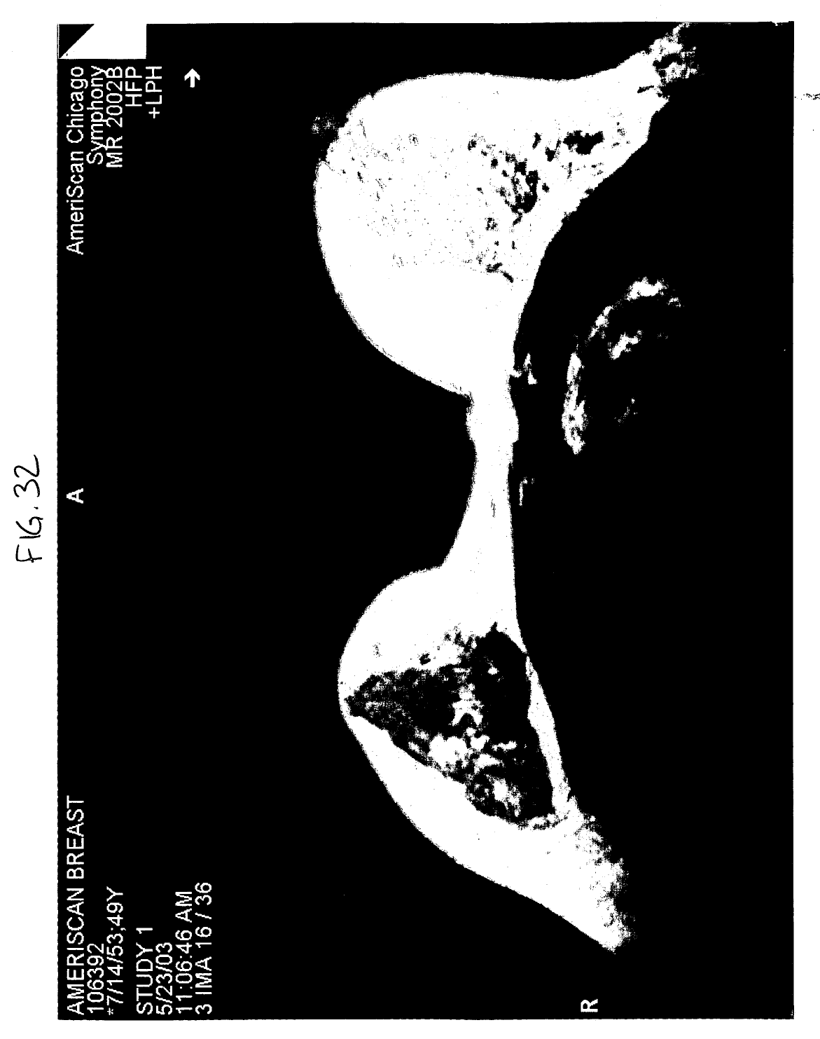

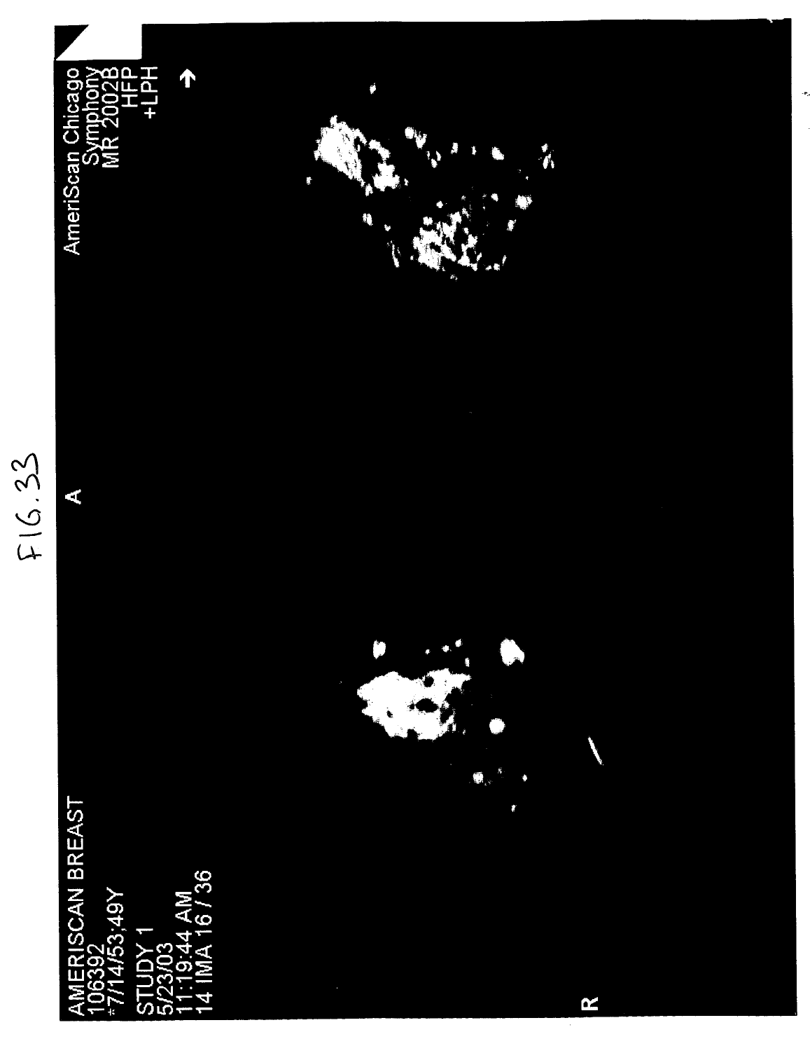

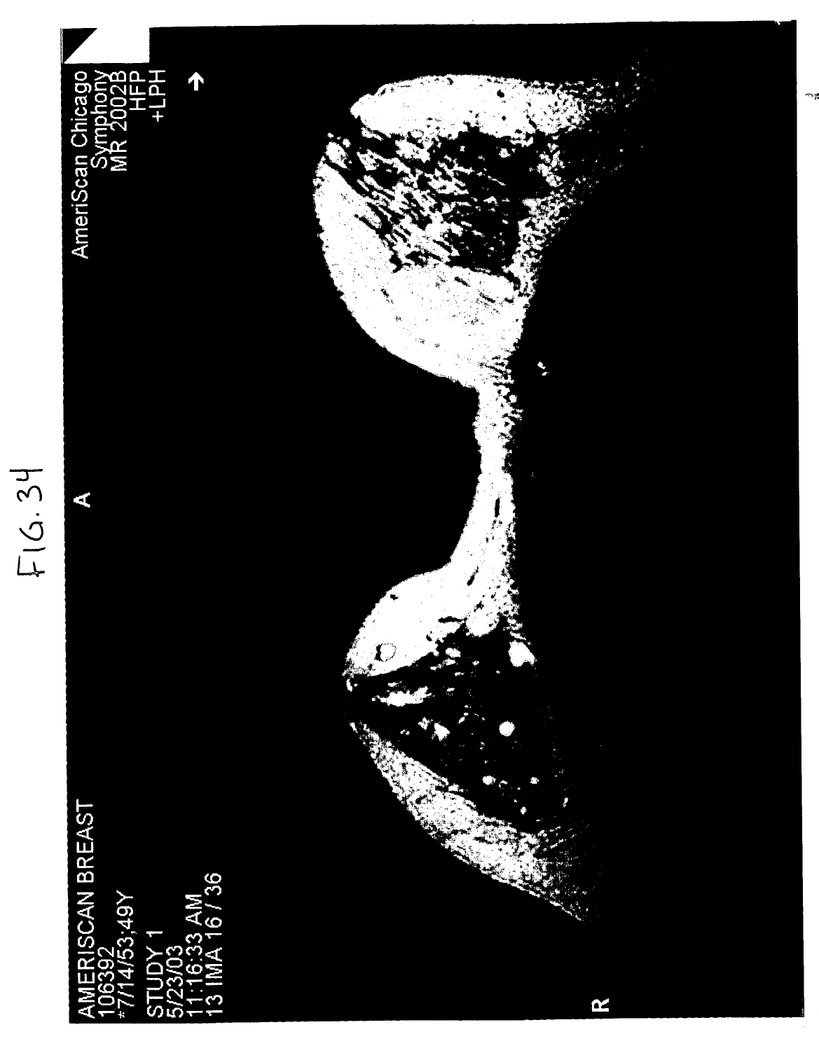

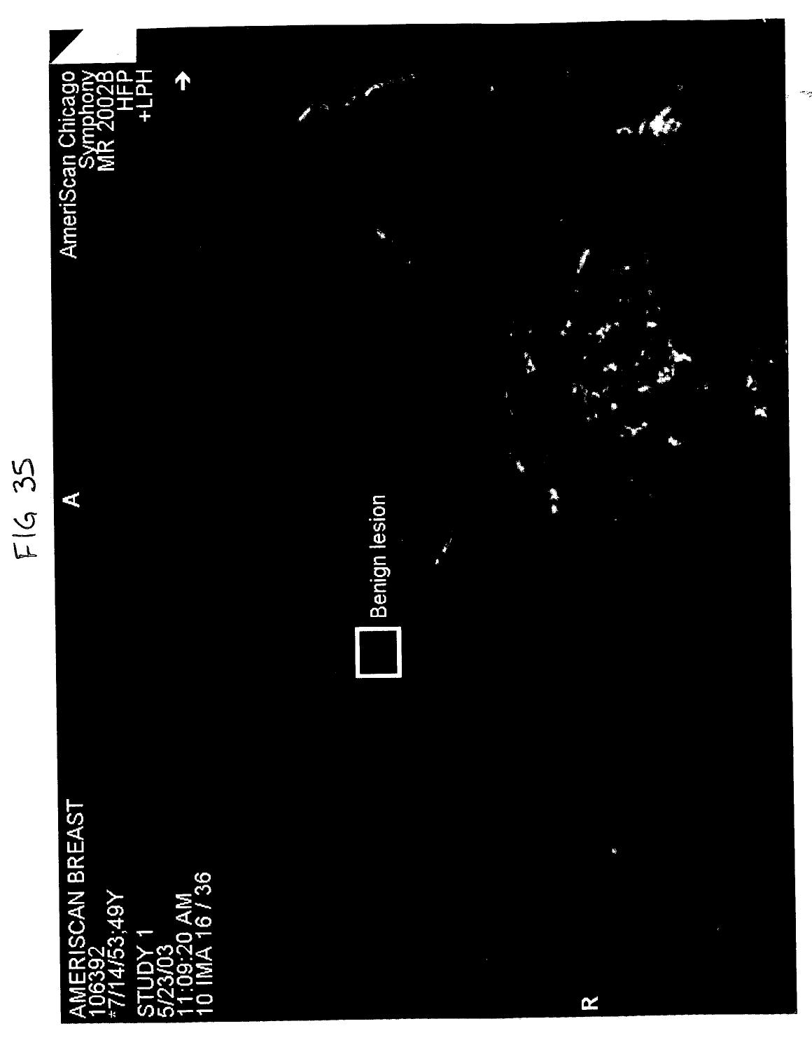

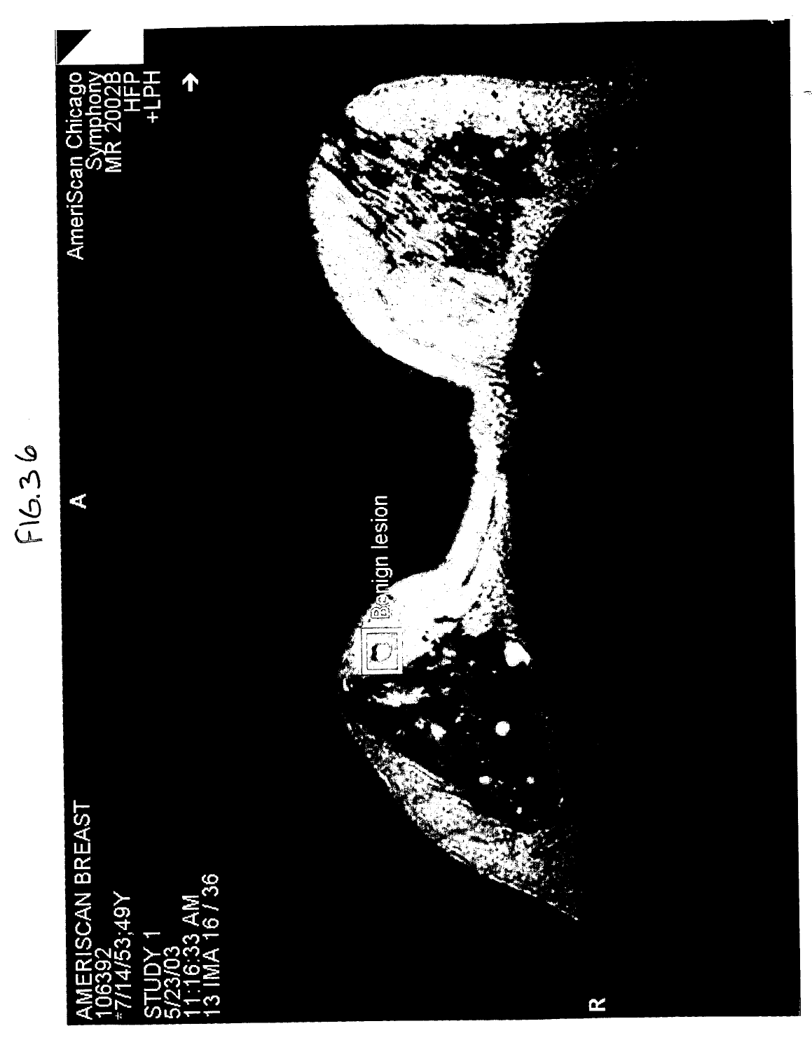

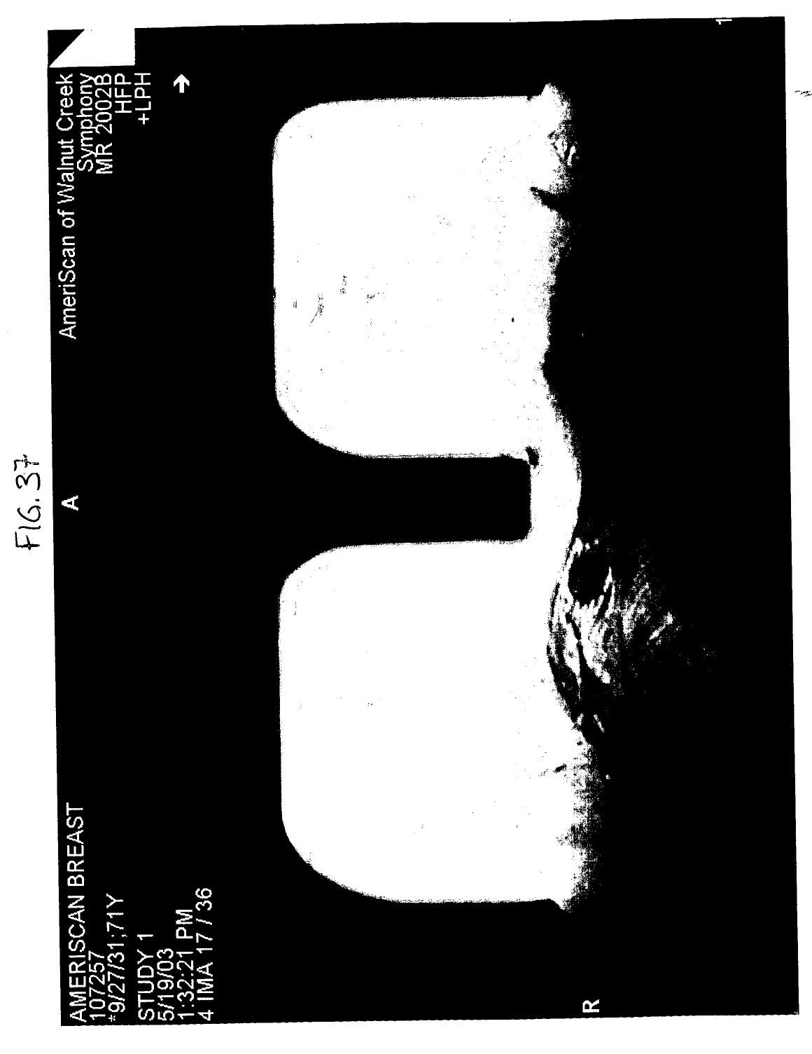

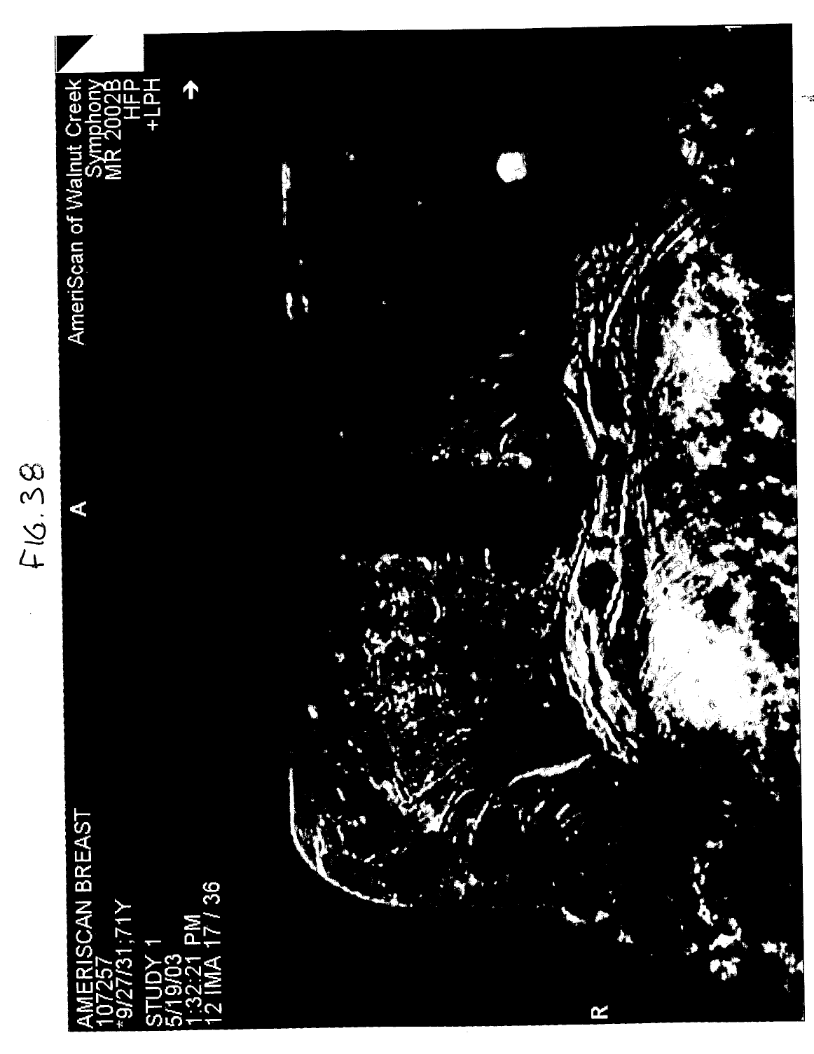

The breasts of an individual were examined using MRI in accordance with the methods of the present invention. FIGS. 22-36 depict sample MRI images of an individual according to the methods of the present invention. This individual had one or more benign lesions. FIG. 22 is a 1 minute post contrast image. FIG. 23 is a 1 minute subtraction image wherein the image data from the pre contrast image was subtracted from that obtained in the 1 minute post contrast image. FIG. 24 is a 2 minute post contrast image. FIG. 25 is a 2 minute subtraction image wherein the image data from the pre contrast image was subtracted from that obtained in the 2 minute post contrast image. FIG. 26 is a 3 minute post contrast image. FIG. 27 is a 3 minute subtraction image wherein the image data from the pre contrast image was subtracted from that obtained in the 3 minute post contrast image. FIG. 28 is a 4 minute post contrast image. FIG. 29 is a 4 minute subtraction image wherein the image data from the pre contrast image was subtracted from that obtained in the 4 minute post contrast image. FIG. 30 is a 5 minute post contrast image. FIG. 31 is a 5 minute subtraction image wherein the image data from the pre contrast image was subtracted from that obtained in the 5 minute post contrast image. FIG. 32 is a pre contrast image. FIG. 33 is a STIR image. FIG. 34 is a T2 weighted image. FIG. 35 demonstrates a location on the 1 minute subtraction image where focal benign nodule is located in the right breast. FIG. 36 demonstrates on the T2 weighted image the location of the same focal benign nodule shown in FIG. 35. FIG. 36 demonstrates a high signal intensity consistent with a benign abnormality. A trained radiologist or other person determined from these images that the scanned individual had one or more benign tumors.

### Example 8

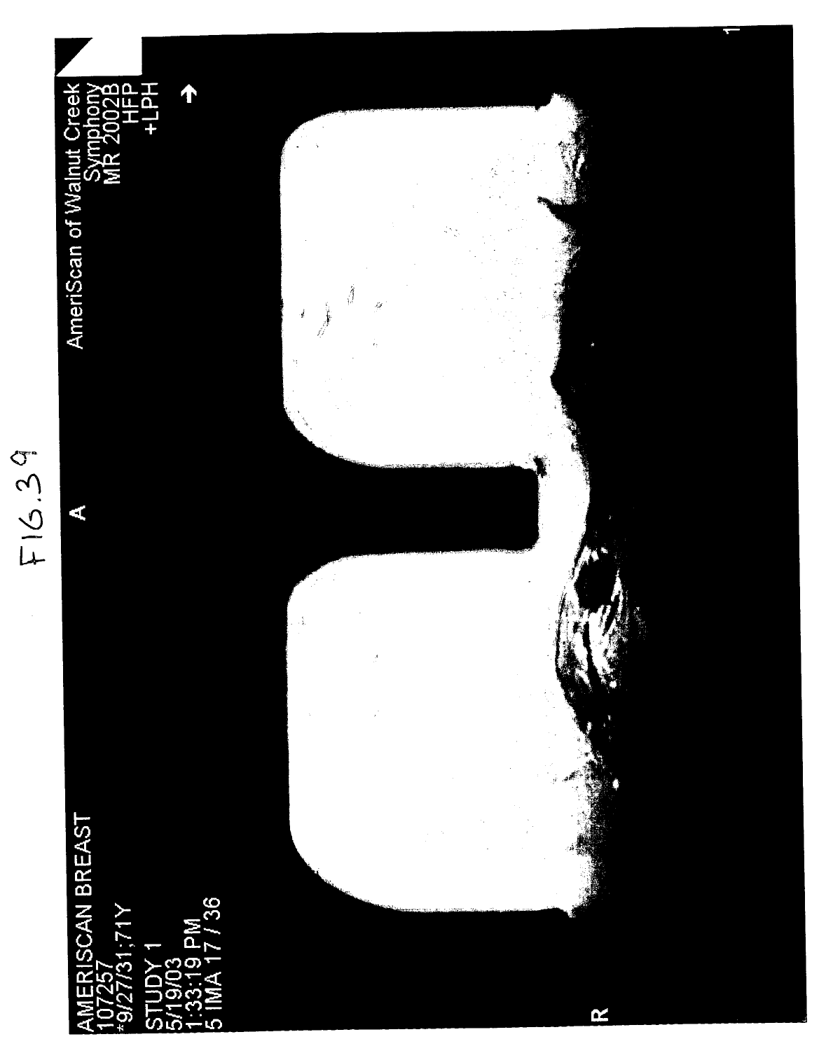

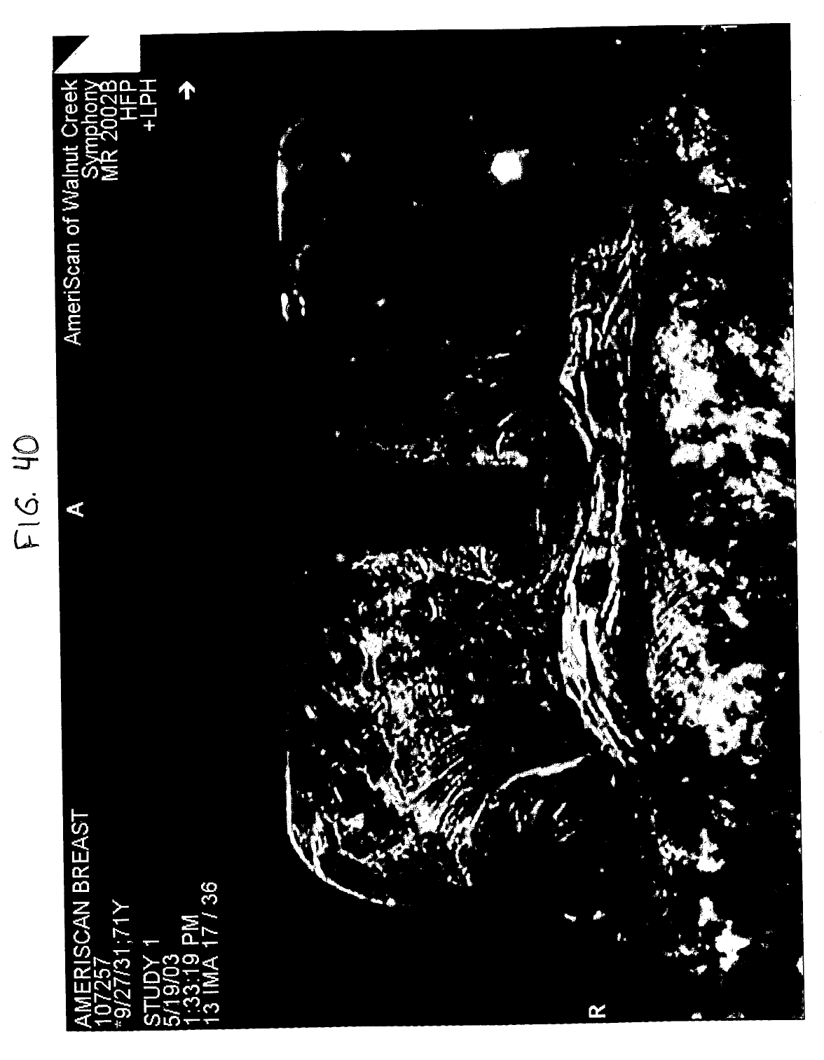

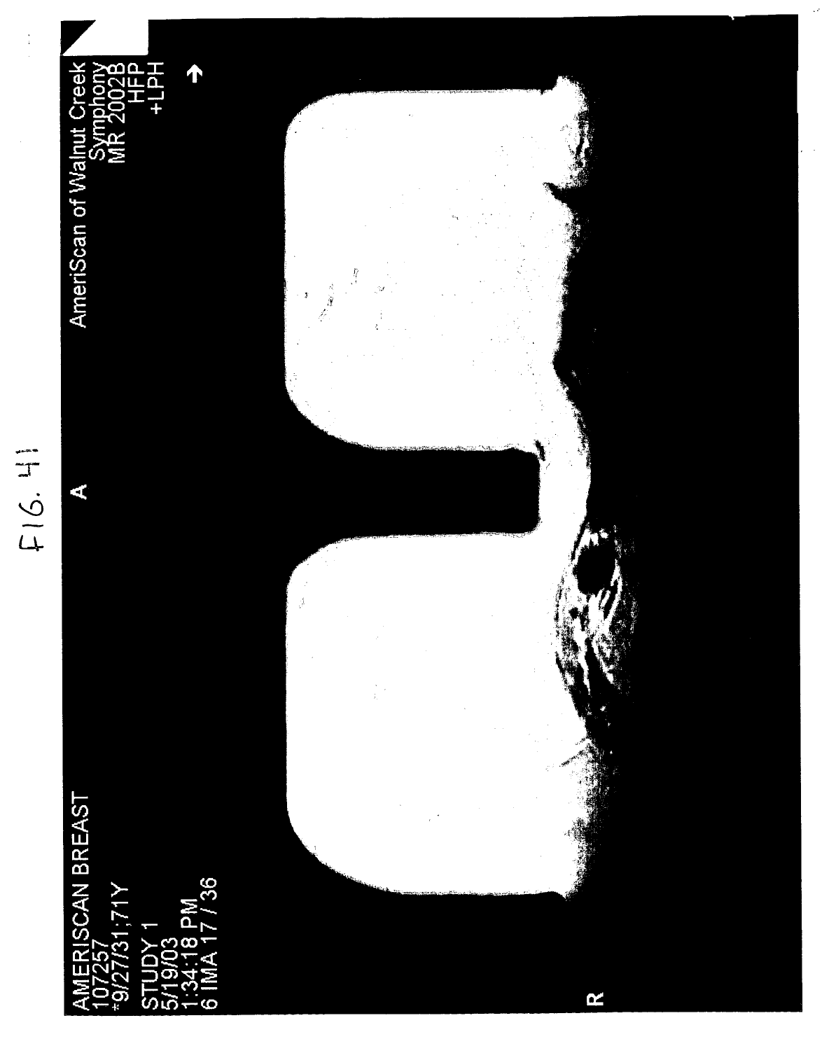

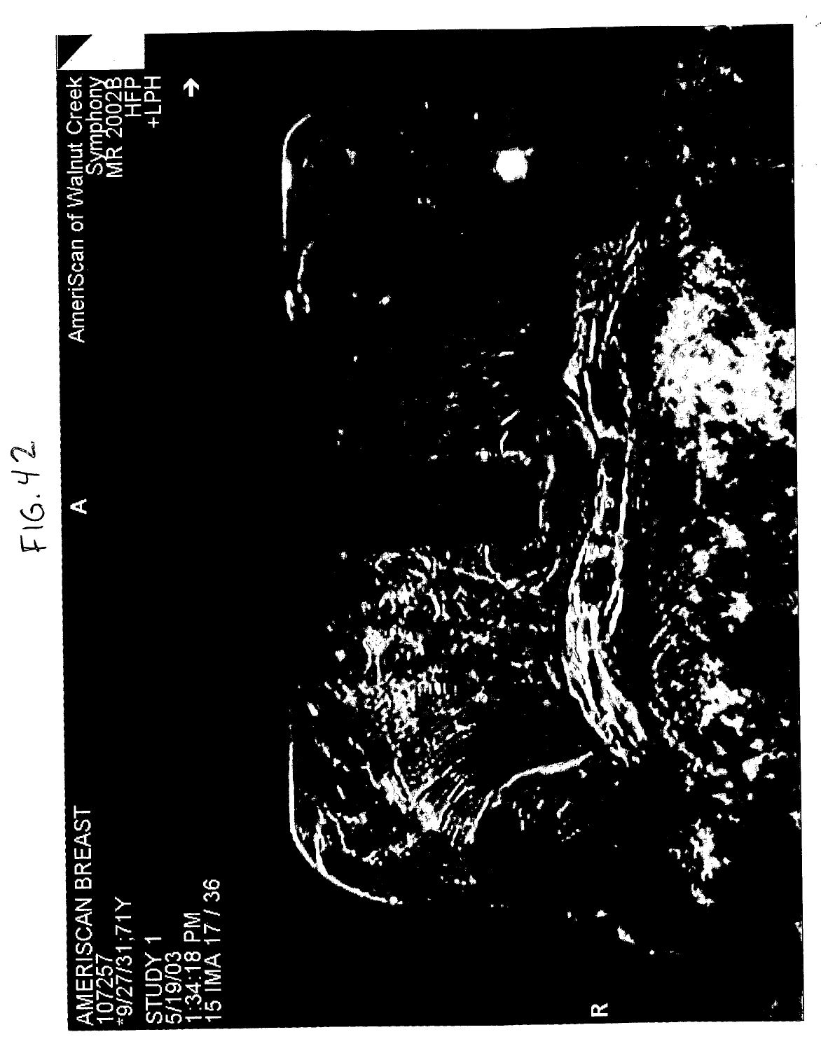

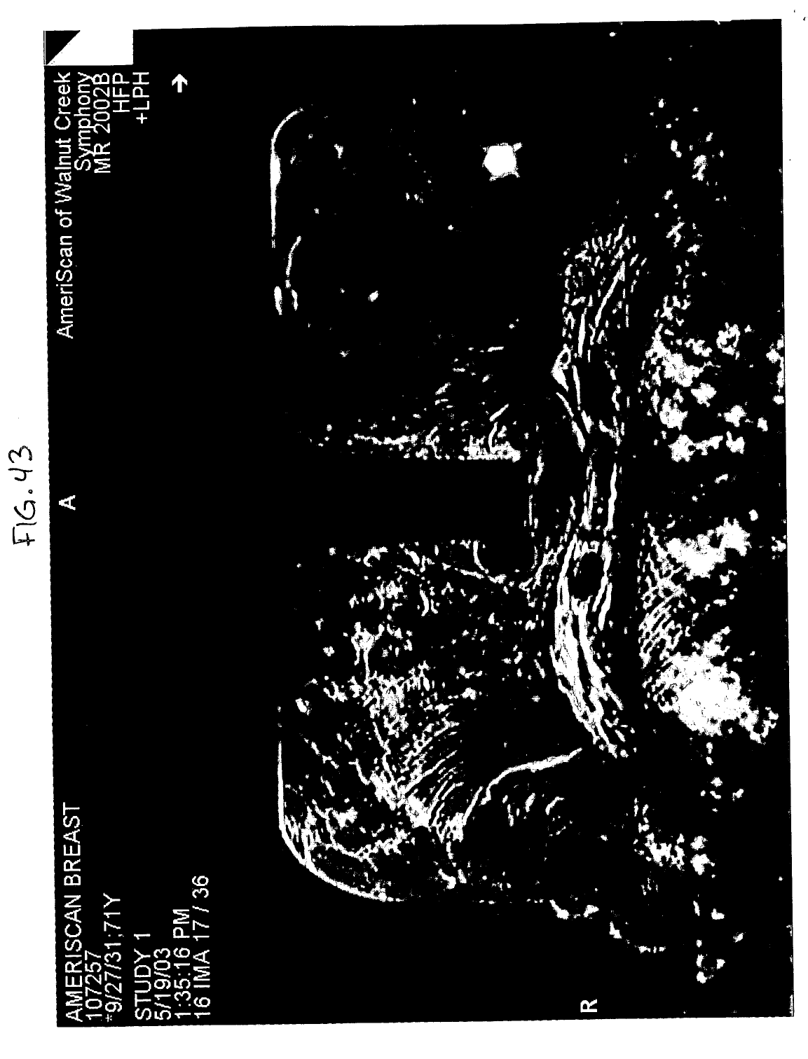

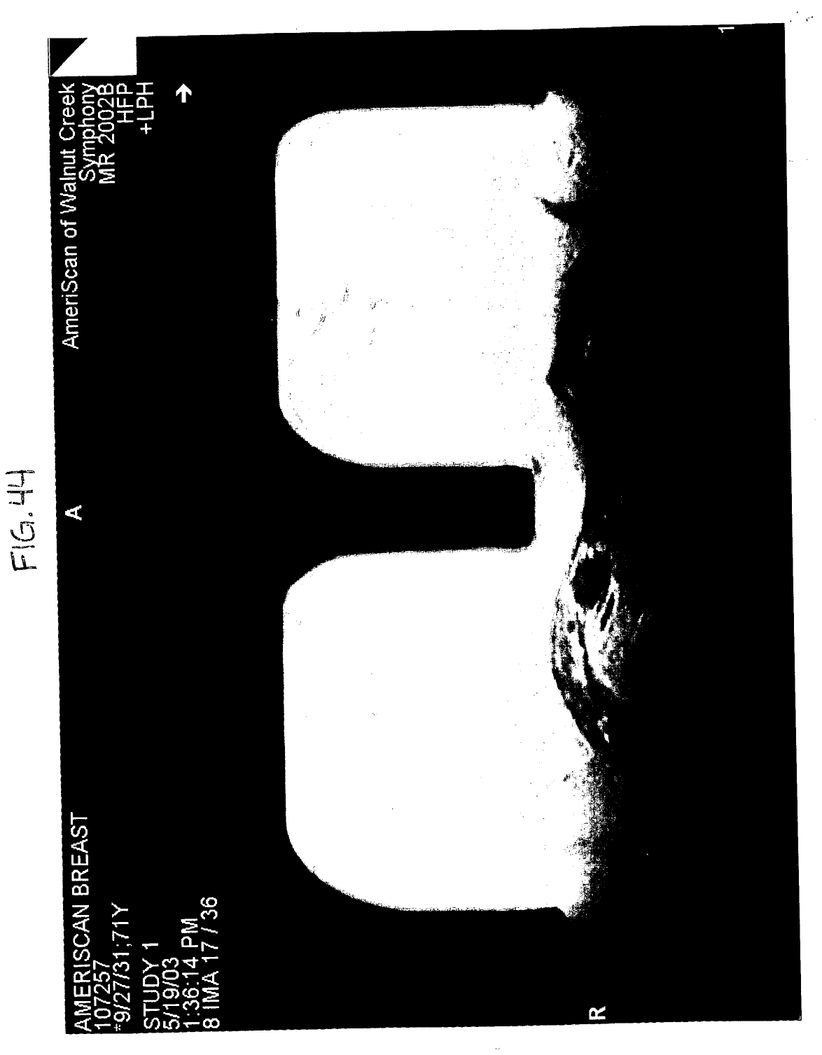

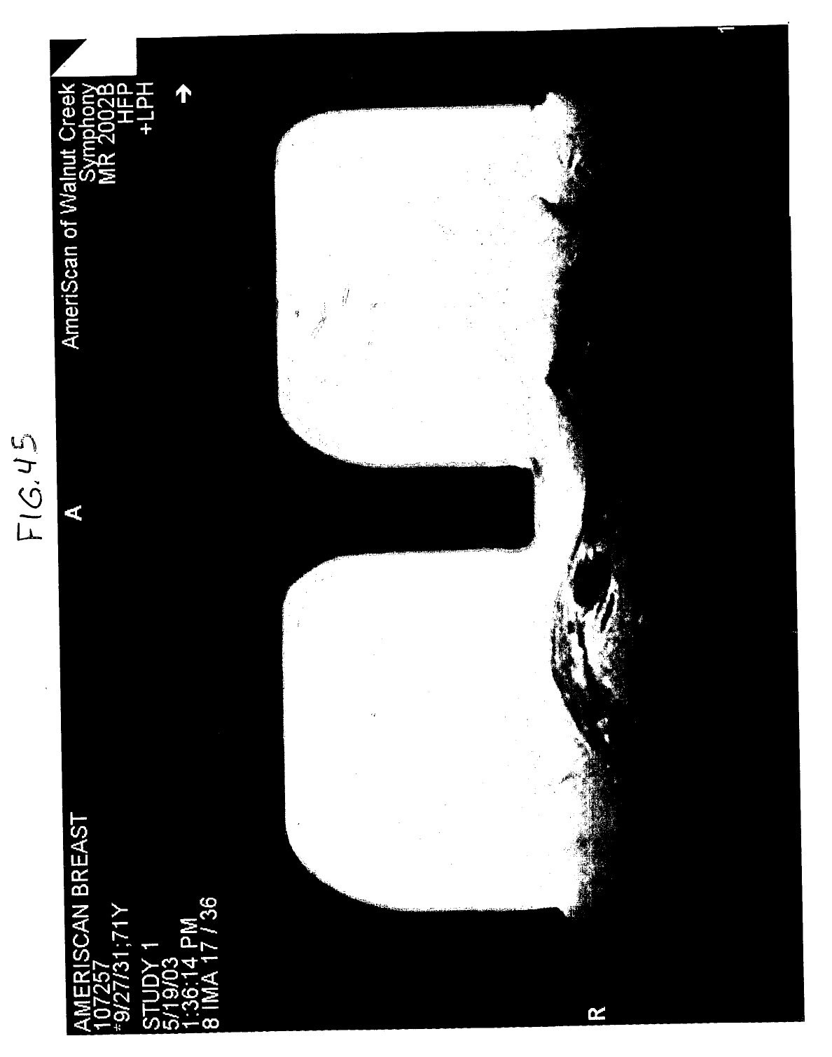

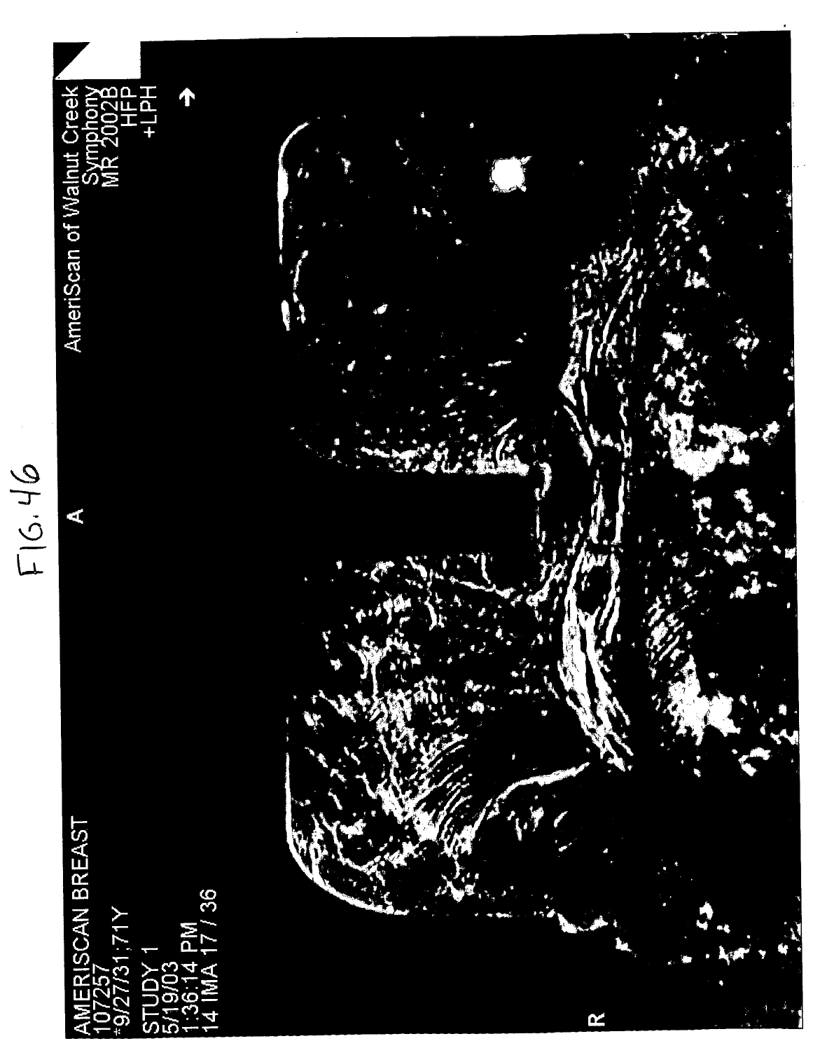

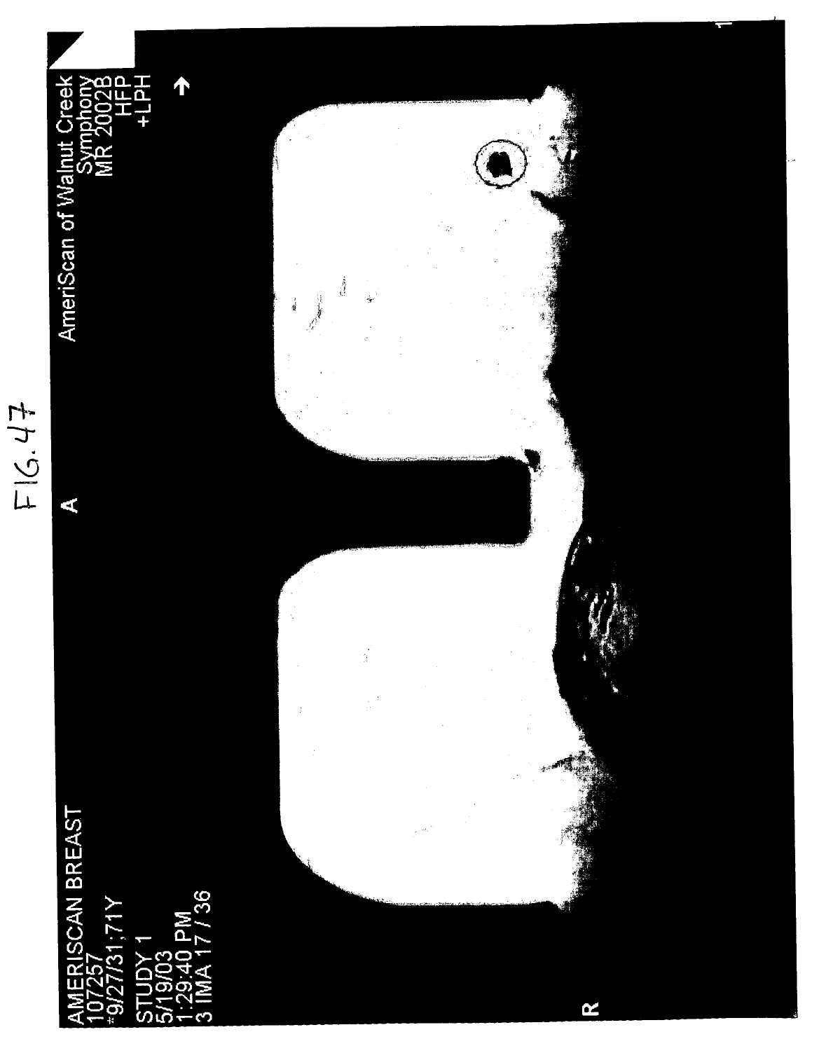

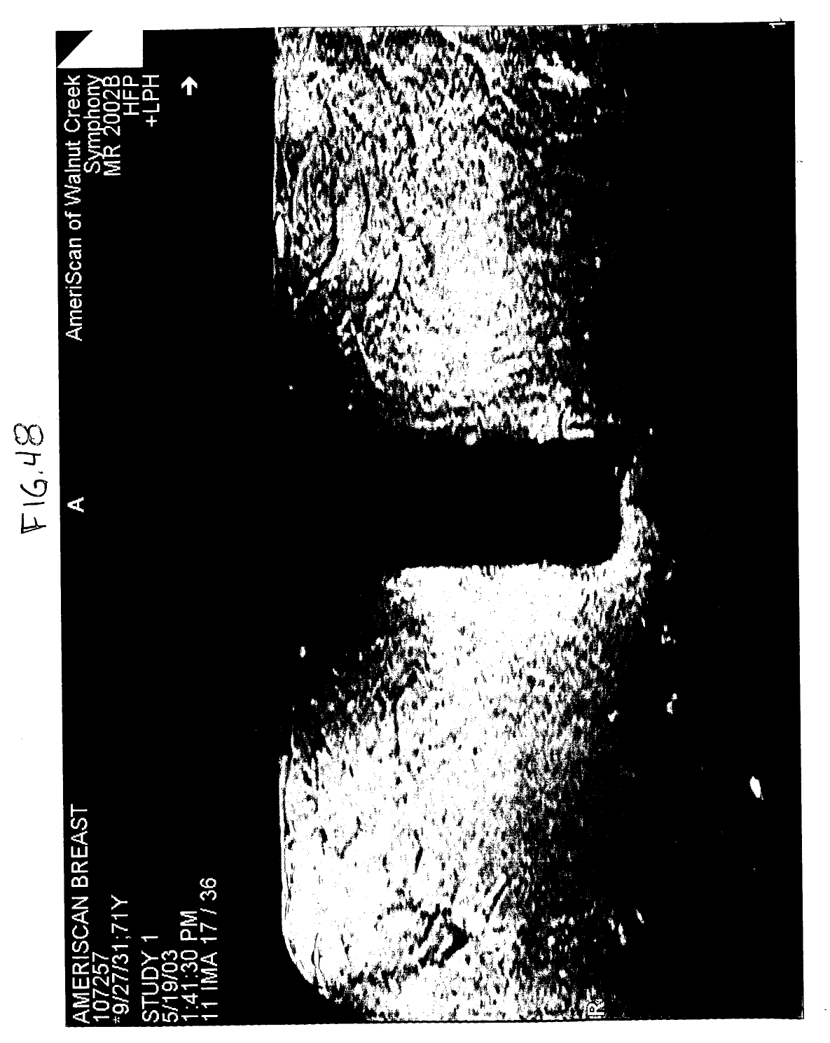

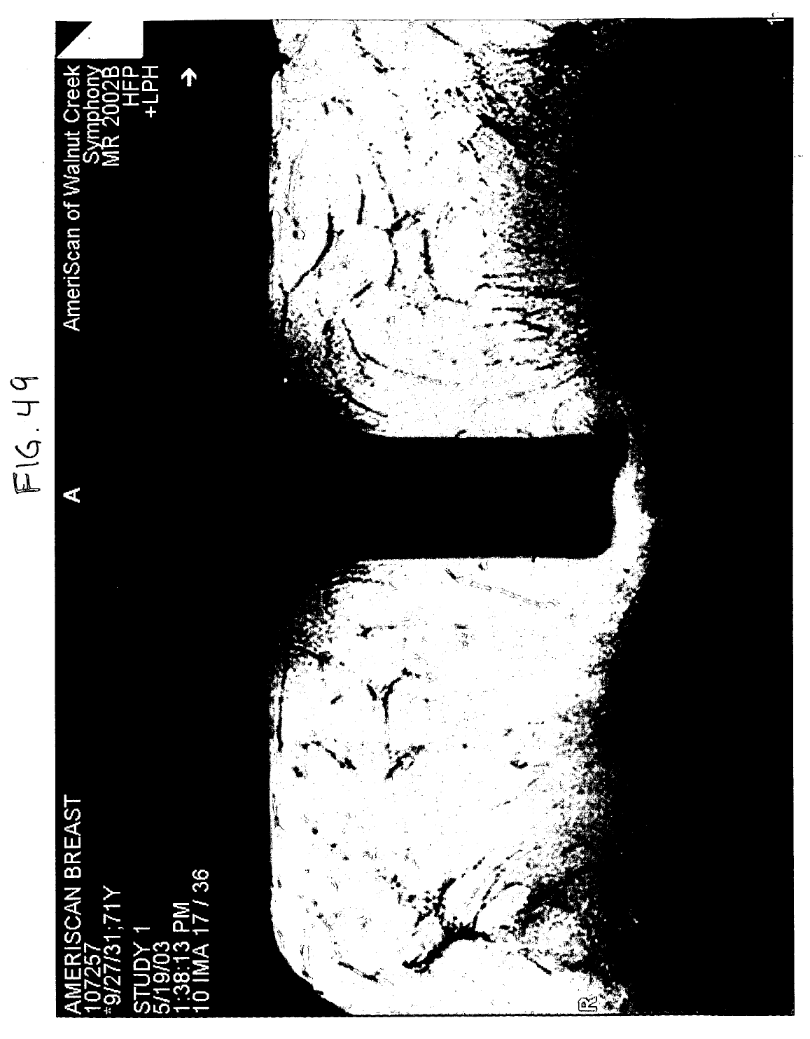

The breasts of an individual were examined using MRI in accordance with the methods of the present invention. FIGS. 37-49 depict sample MRI images of an individual according to the methods of the present invention. This individual had breast cancer. FIG. 37 is a 1 minute post contrast image. FIG. 38 is a 1 minute subtraction image wherein the image data from the pre contrast image was subtracted from that obtained in the 1 minute post contrast image. FIG. 39 is a 2 minute post contrast image. FIG. 40 is a 2 minute subtraction image wherein the image data from the pre contrast image was subtracted from that obtained in the 2 minute post contrast image. FIG. 41 is a 3 minute post contrast image. FIG. 42 is a 3 minute subtraction image wherein the image data from the pre contrast image was subtracted from that obtained in the 3 minute post contrast image. FIG. 43 is a 4 minute post contrast image. FIG. 44 is a 4 minute subtraction image wherein the image data from the pre contrast image was subtracted from that obtained in the 4 minute post contrast image. FIG. 45 is a 5 minute post contrast image. FIG. 46 is a 5 minute subtraction image wherein the image data from the pre contrast image was subtracted from that obtained in the 5 minute post contrast image. FIG. 47 is a pre contrast image. FIG. 48 is a STIR image. FIG. 49 is a T2 weighted image. A trained radiologist or other person determined from these images that the scanned individual had breast cancer.

### Example 9

An MRI spectroscopy examination is performed on an individual, in which the relative concentrations of cellular chemicals and metabolites of the breast tissues are evaluated. MRI spectroscopy can be performed by either single-voxel or a multi-voxel spectroscopy technique at differing spatial resolutions to provide physiologic information about the relative concentrations of the metabolites citrate, creatine, and choline within the breast tissue by measuring the specific resonances for citrate, choline and creatine from small volumes of tissue throughout the breast. The amount of individual resonance present for each metabolite is related to the concentration of these metabolites and changes in these concentrations can be used to identify breast cancer from normal breast tissue and from non-cancerous changes of the breast tissue. Breast cancer will demonstrate significantly higher choline levels and significantly lower citrate levels as compared to normal prostatic tissues and benign changes in the breast. The ratio of these metabolites (e.g., choline/citrate) in regions of cancer have minimal overlap with values from either normal breast tissue or benign breast abnormalities. Also, the physiologic information obtained from MRI spectroscopy may enable assessment of cancer aggressiveness.

While the principles of this invention have been described in connection with specific embodiments, it should be understood clearly that these descriptions are made only by way of example and are not intended to limit the scope of this invention, in any manner. For example, and without limitation, the methodology described herein can be extended to the diagnosis and monitoring of prostate, colon, lung, gall bladder, rectal, pancreatic and oral cancers. Other advantages and features of this invention will become apparent to those skilled in the art.

1. A method for screening normal risk, asymptomatic individuals for breast cancer, comprising
performing MRI on one or more normal risk individuals who are asymptomatic for breast cancer; and
determining from the MRI whether the one or more individuals has indications of breast cancer.

2. The method of claim 1, wherein said method further comprises introducing one or more contrast agents into the one or more individuals before performing MRI.

3. The method of claim 2, wherein the one or more contrast agents comprises paramagnetic metal ions selected from the group consisting of manganese, gadolinium and iron.

4. The method of claim 2, wherein said contrast agent is introduced into a individual by injection.

5. The method of claim 2, wherein said contrast agent is introduced into a individual in an amount of approximately 10-30 mL.

6. The method of claim 2, wherein said contrast agent is introduced into a individual in an amount of approximately 15-25 mL.

7. The method of claim 2, wherein said contrast agent is introduced into a individual in an amount of approximately 15 mL.

8. The method of claim 2, further comprising introducing a physiological saline solution into the individual after introducing the contrast agent, in approximately the same volume as the contrast agent.

9. A method of detecting breast cancer in normal risk, asymptomatic individuals, comprising screening normal risk individuals who are asymptomatic for breast cancer, wherein said screening comprises
performing MRI on one or more individuals; and
determining from the MRI whether the one or more individuals has indications of breast cancer.

10. The method of claim 9, wherein said method further comprises introducing one or more contrast agents into the one or more individuals before performing MRI.

11. The method of claim 10, wherein the one or more contrast agents comprises paramagnetic metal ions selected from the group consisting of manganese, gadolinium and iron.

12. The method of claim 10, wherein said contrast agent is introduced into a individual by injection.

13. The method of claim 10, wherein said contrast agent is introduced into a individual in an amount of approximately 10-30 mL.

14. The method of claim 10, wherein said contrast agent is introduced into a individual in an amount of approximately 15-25 mL.

15. The method of claim 10, wherein said contrast agent is introduced into a individual in an amount of approximately 15 mL.

16. The method of claim 10, further comprising introducing a physiological saline solution into the individual after introducing the contrast agent, in approximately the same volume as the contrast agent.

17. A method of screening a normal risk, asymptomatic individual for breast cancer comprising
obtaining a baseline pre-contrast MRI of an entire breast of a individual;
administering one or more contrast agents to the individual in an amount of approximately 0.07-0.13 mmol/kg body weight of the individual;
performing a dynamically enhanced MRI with T1 weighting of the breast over a period of approximately 2 to 10 minutes at intervals of approximately every 5 to 90 seconds;
plotting a degree of percentage of MRI signal enhancement over time; and
differentiating regions of cancer growth from normal breast tissue based on the degree of enhancement.

18. The method of claim 17, wherein the contrast agent is administered to the individual in an amount of approximately 0.085-0.115 mmol/kg body weight of the individual.

19. The method of claim 17, wherein the contrast agent is administered to the individual in an amount of approximately 0.1 mmol/kg body weight of the individual.

20. The method of claim 17, wherein the contrast agent is administered at a bolus rate of approximately 1 to 3 cc/sec.

21. The method of claim 17, wherein the contrast agent is administered at a bolus rate of approximately 2 cc/sec.

22. The method of claim 17, wherein the one or more contrast agents comprises paramagnetic metal ions selected from the group consisting of manganese, gadolinium and iron.

23. The method of claim 22, wherein the one or more contrast agents comprises gadolinium.

24. The method of claim 17, wherein the method is performed on both of an individual's breasts substantially simultaneously.

25. The method of claim 17, further comprising performing T2 weighted imaging of the breast.

26. The method of claim 17, further comprising performing proton density weighted imaging of the breast.

27. The method of claim 1, wherein said method comprises performing MRI spectroscopy on the one or more individuals.

28. A method for screening normal risk, asymptomatic individuals for breast cancer, comprising
performing MRI spectroscopy on one or more normal risk individuals who are asymptomatic for breast cancer; and
determining if the one or more individuals have indications of breast cancer.

29. A method of screening an individual for breast cancer comprising
obtaining a MRI spectroscopy of at least one breast of a normal risk individual who is asymptomatic for breast cancer;
determining relative concentrations of metabolites within breast tissue of the at least one breast by measuring specific resonances of said metabolites from tissue throughout the at least one breast;
determining ratios of said metabolites in the breasts; and
differentiating regions of cancer growth from normal breast tissue based on the ratios.

30. The method of claim 29, wherein the metabolites comprise one or more metabolites selected from the group consisting of citrate, creatine and choline.
